(19) United States
(12) Patent Application Publication
(10) Pub. No.: US 2024/0252779 A1
(43) Pub. Date: Aug. 1, 2024

(54) A GAS FLOW CONTROL STRUCTURE FOR A RESPIRATORY THERAPY SYSTEM

(71) Applicant: RESMED PTY LTD, Bella Vista, New South Wales (AU)

(72) Inventors: Shiva Kumar SHANMUGA SUNDARA, Sydney (AU); Rahul KHERA, Sydney (AU)

(21) Appl. No.: 18/577,185

(22) PCT Filed: Jul. 7, 2022
(86) PCT No.: PCT/AU2022/050709

§ 371 Date: Jan. 5, 2024

(30) Foreign Application Priority Data
Jul. 9, 2021 (AU) 2021902102

(51) Int. Cl. A61M 16/20; A61M 16/06; A61M 16/16

(52) U.S. Cl. CPC A61M 16/209; A61M 16/0616; A61M 16/0683; A61M 16/16

(57) ABSTRACT

A gas flow control structure for a respiratory therapy system, which may be located on a patient interface, may function as both an anti-asphyxia valve (AAV) and a vent. The gas flow control structure may comprise: a base member; a vent member comprising a vent opening through which a flow of gas can pass from a pressurised first volume to an ambient second volume; an AAV opening formed between the base member and the vent member; and an AAV member. The AAV member may be a flexible, resilient membrane substantially annular in shape when projected on to a plane. The AAV member may be movable between: an activated configuration in which gas can flow between the first and second volumes through the AAV opening; and a deactivated configuration in which the AAV member forms a seal with the vent member so gas is blocked from flowing through the AAV opening.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

### 1.2.2 Therapies

Various respiratory therapies, such as Continuous Positive Airway Pressure (CPAP) therapy. Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

### 1.2.2.2 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

Systems allowing for the supply of supplementary oxygen may have an oxygen diverter valve (ODV). The ODV can be used to prevent oxygen flowing upstream into the RPT device when there is no pressurised flow of air. In some systems the ODV is located downstream of the RPT device and upstream of the oxygen supply, for example at a point along the air circuit. Typically the ODV is located close to the RPT device.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use. An assembly of straps comprised as part of a patient interface may be referred to as headgear.

### 1.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as a “tube down” configuration.

Some patients find such interfaces to be unsightly or to create a feeling of claustrophobia and are consequently deterred from wearing them, reducing patient compliance. Additionally, conduits connecting to an interface at the front of a patient's face may sometimes be vulnerable to becoming tangled up in bed clothes.

1.2.3.1.4 Pressurised Air Conduit used for Positioning/Stabilising the Seal-Forming Structure

An alternative type of treatment system which seeks to address these problems comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

Patient interfaces incorporating headgear tubing may provide some advantages, for example avoiding a conduit connecting to the patient interface at the front of a patient's face, which may be unsightly and obtrusive. However, it is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 1.2.3.6 Vent Technologies

Some forms of treatment systems may include a vent to allow the washout of exhaled carbon dioxide. The vent may allow a flow of gas from an interior space of a patient interface. e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2:2007, 10 cmH2O pressure at 1 m)

| | | A-weighted | | A-weighted | | |
|---|---|---|---|---|---|---|
| | | sound power | | sound pressure | | |
| | Mask | level dB(A) | | dB(A) | | Year |
| Mask name | type | (uncertainty) | | (uncertainty) | | (approx.) |
| Glue-on (*) | nasal | 50.9 | | 42.9 | | 1981 |
| ResCare | nasal | 31.5 | | 23.5 | | 1993 |
| standard (*) | | | | | | |
| ResMed | nasal | 29.5 | | 21.5 | | 1998 |
| MirageTM (*) | | | | | | |
| ResMed | nasal | 36 | (3) | 28 | (3) | 2000 |
| UltraMirageTM | | | | | | |
| ResMed | nasal | 32 | (3) | 24 | (3) | 2002 |
| Mirage | | | | | | |
| ActivaTM | | | | | | |
| ResMed | nasal | 30 | (3) | 22 | (3) | 2008 |
| Mirage | | | | | | |
| MicroTM | | | | | | |
| ResMed | nasal | 29 | (3) | 22 | (3) | 2008 |
| MirageTM | | | | | | |
| SoftGel | | | | | | |
| ResMed | nasal | 26 | (3) | 18 | (3) | 2010 |
| MirageTM FX | | | | | | |
| ResMed | nasal | 37 | | 29 | | 2004 |
| Mirage | pillows | | | | | |
| SwiftTM (*) | | | | | | |
| ResMed | nasal | 28 | (3) | 20 | (3) | 2005 |
| Mirage | pillows | | | | | |
| SwiftTM II | | | | | | |
| ResMed | nasal | 25 | (3) | 17 | (3) | 2008 |
| Mirage | pillows | | | | | |
| SwiftTM LT | | | | | | |
| ResMed AirFit | nasal | 21 | (3) | 13 | (3) | 2014 |
| P10 | pillows | | | | | |

(*) one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

An aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

One aspect of the present technology relates to a gas flow control structure for use in a respiratory therapy system. The gas flow control structure may be configured to function as a vent to allow for the washout of exhaled gases, e.g. carbon dioxide, from the respiratory therapy system. The gas flow control structure may additionally be configured to function as an anti-asphyxia valve (AAV) to provide a flow path between the patient's airways and the ambient atmosphere in the event of a cessation of the supply of breathable gas, or a drop in pressure of the supply of breathable gas to below a certain pressure level.

An aspect of one form of the present technology is a gas flow control structure for a respiratory therapy system configured to function as both an anti-asphyxia valve (AAV) and a vent, wherein the gas flow control structure separates a first volume that is pressurised during use and a second volume that is the surrounding ambient air, the gas flow control structure comprising:
a base member;
a vent member comprising at least one vent opening through which a flow of gas can pass from the first volume to the second volume during use;
an AAV opening formed between the base member and the vent member; and
an AAV member mounted to the base member, wherein the AAV member is a flexible, resilient membrane which is substantially annular in shape when projected on to a plane, the annular shape of the AAV member forming a central hole,
wherein the AAV member is movable between: an activated configuration in which gas can flow between the first volume and the second volume through the AAV opening; and a deactivated configuration in which the AAV member forms a seal with a portion of the vent member so gas is blocked from flowing through the AAV opening,
wherein the AAV member is biased towards the activated configuration such that, when the first volume contains gas having a pressure that is below an activation pressure, the AAV member is in the activated configuration, and, when the first volume contains gas having a pressure that is above a deactivation pressure, the AAV member is in the deactivated configuration;
wherein the AAV member is arranged such that gas from the first volume flows through the central hole in the AAV member when passing to the second volume through the at least one vent opening.

In examples: a) the AAV member comprises: a first segment with a first end and an opposite second end, wherein the first end of the first segment is rotatably connected to a mounted part of the AAV member at a first rotatable connection, wherein the mounted part of the AAV member is mounted to the base member; and a second segment with a first end and an opposite second end, wherein the first end of the second segment is rotatably connected to the second end of the first segment at a second rotatable connection; b) the AAV member further comprises a foot portion that is rotatably connected to the second end of the second segment at a third rotatable connection; c) the foot portion contacts a portion of the base member in the activated configuration; d) the base member comprises an annular flange that the foot portion contacts in the activated configuration; e) the foot portion forms the seal with the portion of the vent member in the deactivated configuration; f) a substantially flat plane of the foot portion forms the seal with the portion of the vent member in the deactivated configuration; g) the vent member comprises an annular flange that the foot portion forms the seal with in the deactivated configuration; h) the annular flange of the vent member and the annular flange of the base member are substantially similar in size and are offset along a central axis of the gas flow control structure; i) in the activated configuration the angle between the first segment and the second segment at the second rotatable connection is between 45 and 80°; j) in the activated configuration the angle between the first segment and the second segment at the second rotatable connection is between 60 and 65°; k) in the deactivated configuration angle between the first segment and the second segment at the second rotatable connection is greater than 90°; l) in the deactivated configuration the angle between the first segment and the second segment at the second rotatable connection is between 90° and 110°; m) further comprising a diffuser positioned adjacent the at least one vent opening so the flow of gas through the at least one vent opening passes through the diffuser prior to reaching the second volume; n) further comprising a cover member, the cover member connects to the vent member and to the base member, and the cover member comprises one or more cover openings through which gas flows after passing through the AAV opening and after passing through the vent opening; o) the cover member is substantially dome-shaped; p) the vent member connects directly to the base member and further comprises one or more ambient openings through which gas flows after passing through the AAV opening; q) further comprising a diffuser cover to cover the diffuser, a gap between the vent member and the diffuser cover allows gas to flow from the diffuser to the second volume; and r) a neutral configuration of the AAV member is different to the activated configuration.

In some forms the AAV member is integrally formed in one piece. In examples: the first, second, and/or third rotatable connections are formed by bends and/or curves in the AAV member.

In another aspect of the technology there is provided a patient interface. The patient interface may be suitable for use in a respiratory therapy system for delivering breathable gas at a therapeutic pressure of at least 6 cmH₂O above ambient air pressure for breathing by a patient.

An aspect of one form of the technology is a patient interface comprising:
a plenum chamber pressurisable to a therapeutic pressure of at least 6 cmH2O above ambient air pressure, said plenum chamber including a plenum chamber inlet port sized and structured to receive a flow of gas at the therapeutic pressure for breathing by a patient;
a seal-forming structure constructed and arranged to form a seal with a region of the patient's face surrounding an entrance to the patient's airways, said seal-forming structure having a hole therein such that the flow of gas at said therapeutic pressure is delivered to at least an entrance to the patient's nares, the seal-forming structure constructed and arranged to maintain said therapeutic pressure in the plenum chamber throughout the patient's respiratory cycle in use; and
a gas flow control structure as described in any of the aspects of the technology above, wherein the gas flow control structure allows a continuous flow of gas exhaled by the patient from an interior of the plenum chamber to ambient, said gas flow control structure being sized and shaped to maintain the therapeutic pressure in the plenum chamber in use when the AAV member is in the deactivated configuration, and wherein the gas flow control structure allows the patient to inhale and exhale air from ambient when the flow of gas at the therapeutic pressure is not being supplied to the plenum chamber.

In an example the gas flow control structure is located in a wall of the plenum chamber.

An aspect of one form of the technology is a respiratory therapy system comprising:
a patient interface as described in any of the other aspects of the technology above, and
at least one air circuit for supplying the flow of gas at the therapeutic pressure to the patient interface.

In an example the AAV member is configured to allow gas to flow between the plenum chamber and the air circuit in both the activated configuration and deactivated configuration.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

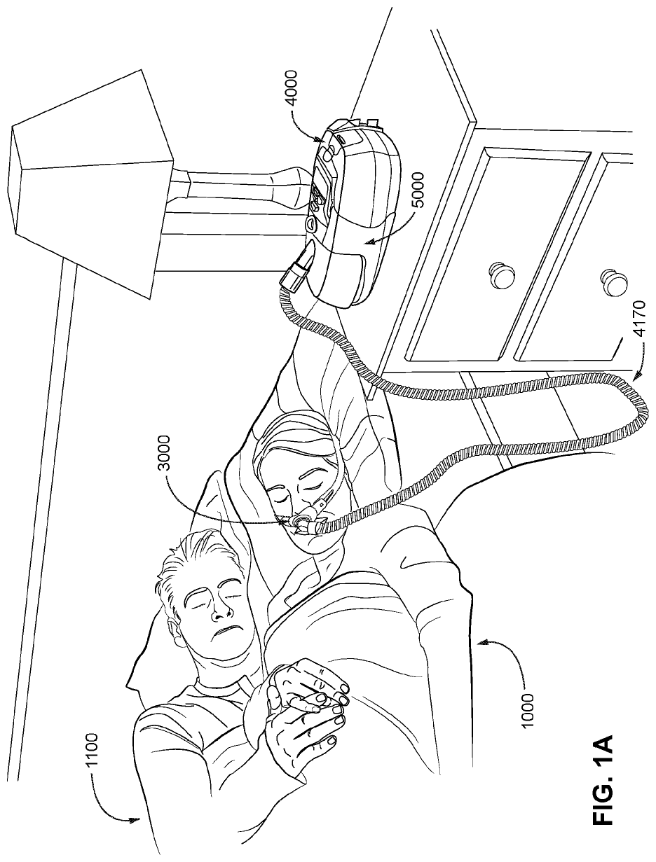

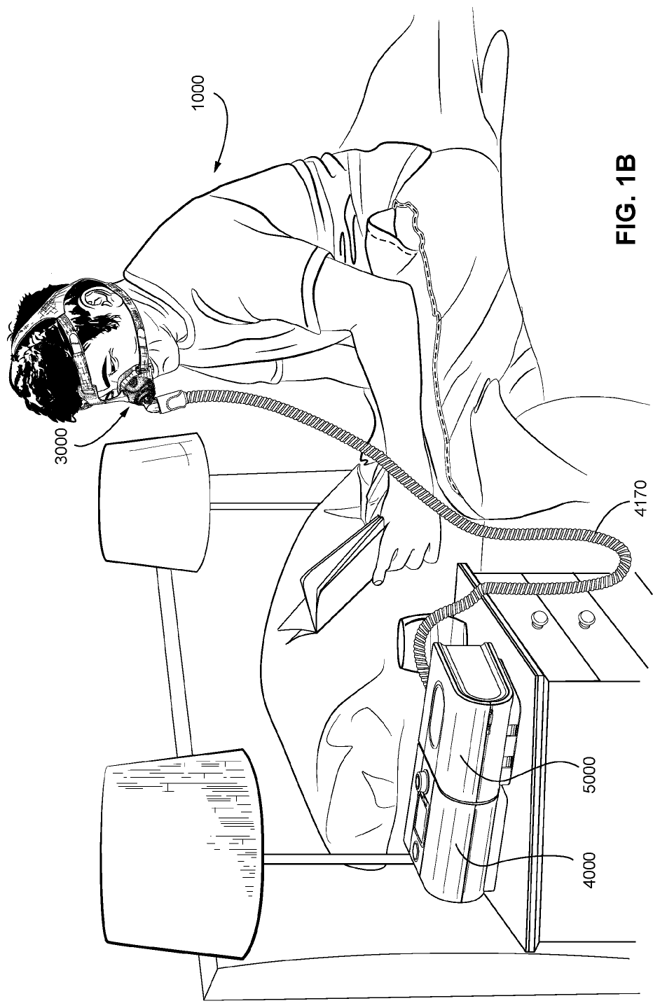

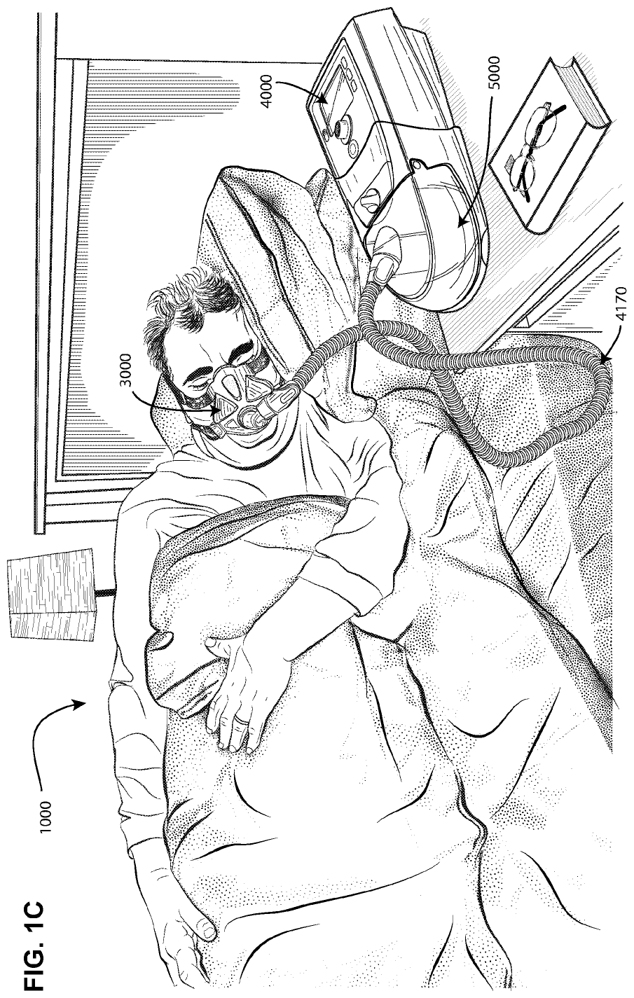

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

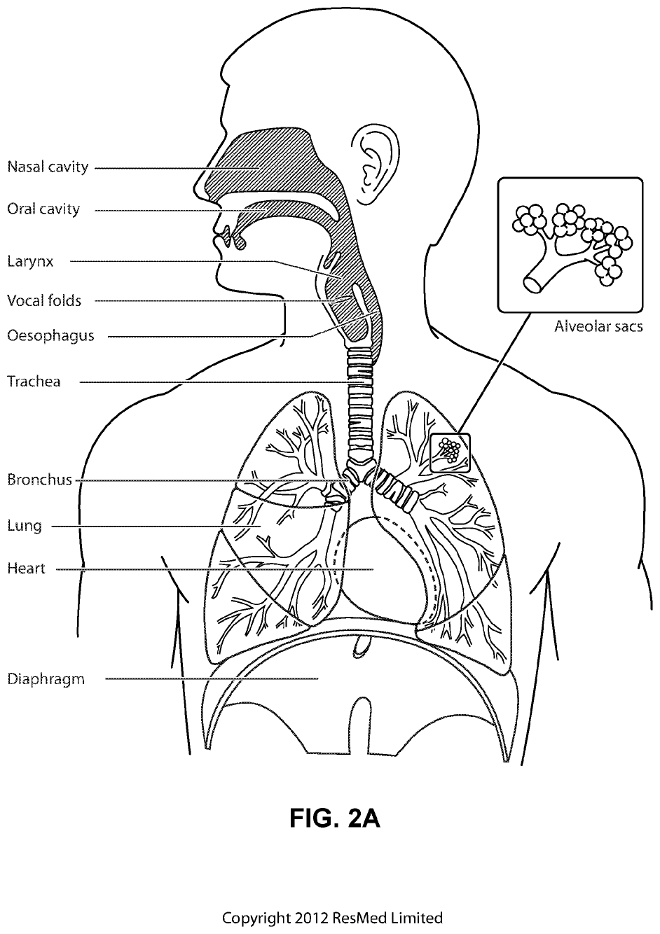

FIG. 2A shows an overview of a human respiratory system including the nasal and oral cavities, the larynx, vocal folds, oesophagus, trachea, bronchus, lung, alveolar sacs, heart and diaphragm.

### 3.3 Patient Interface

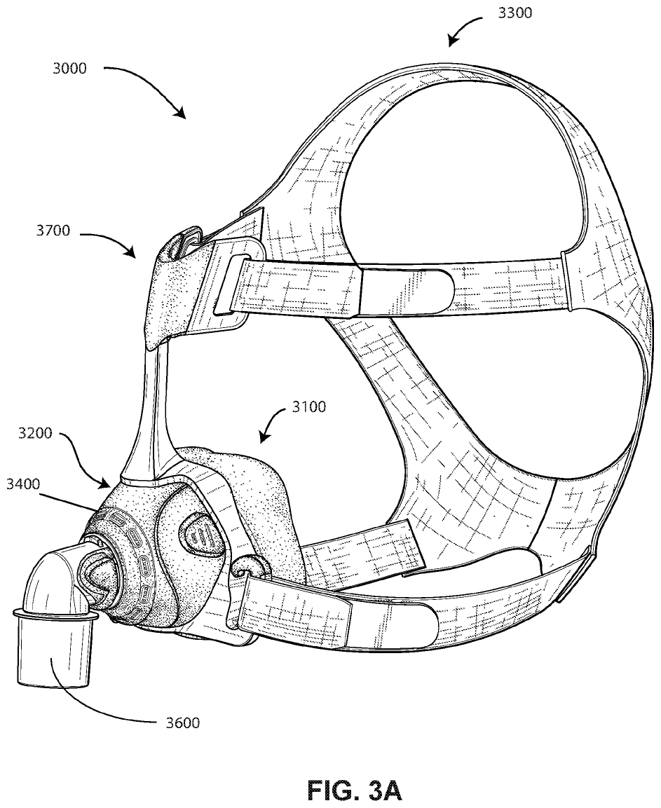

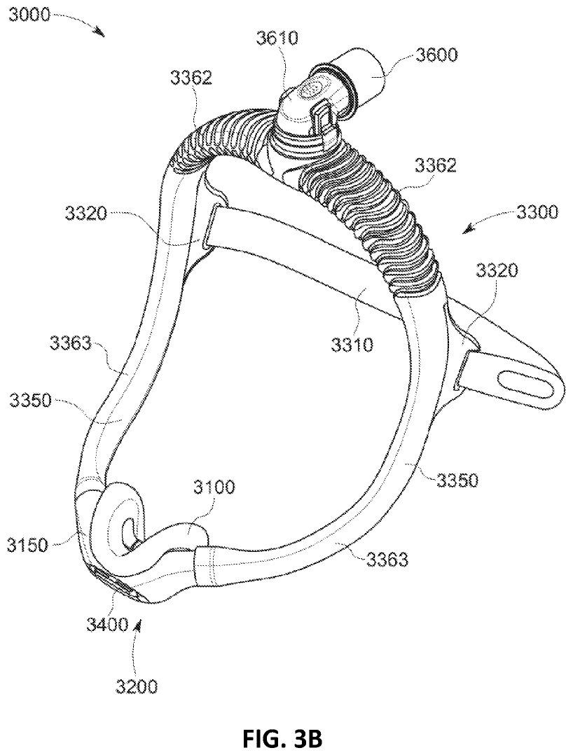

FIG. 3A shows a patient interface in the form of a nasal mask in accordance with one form of the present technology.

FIG. 3B shows a patient interface in the form of a mask having conduit headgear in accordance with one form of the present technology.

### 3.4 RPT Device

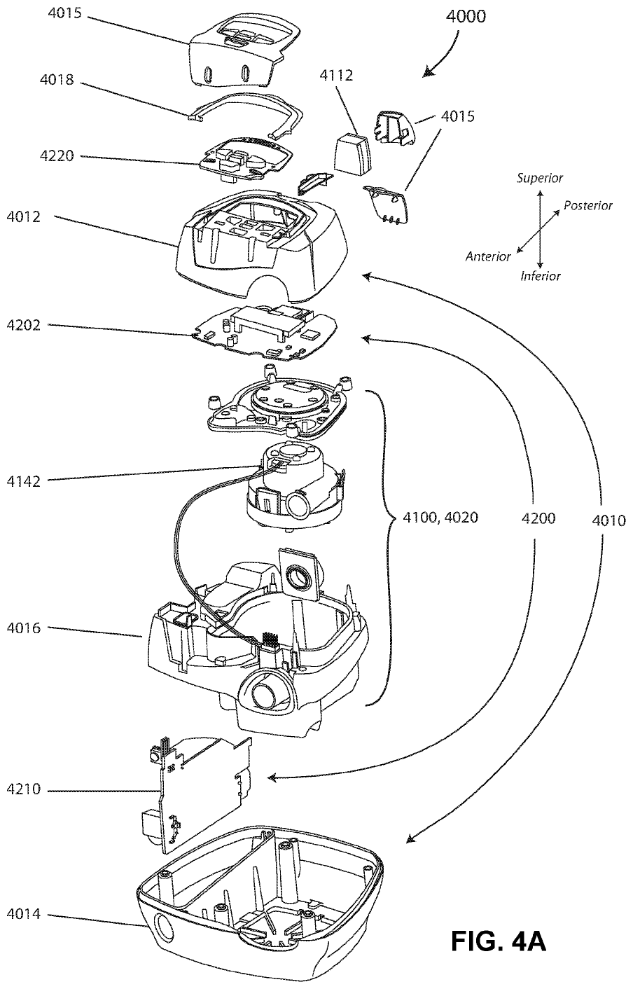

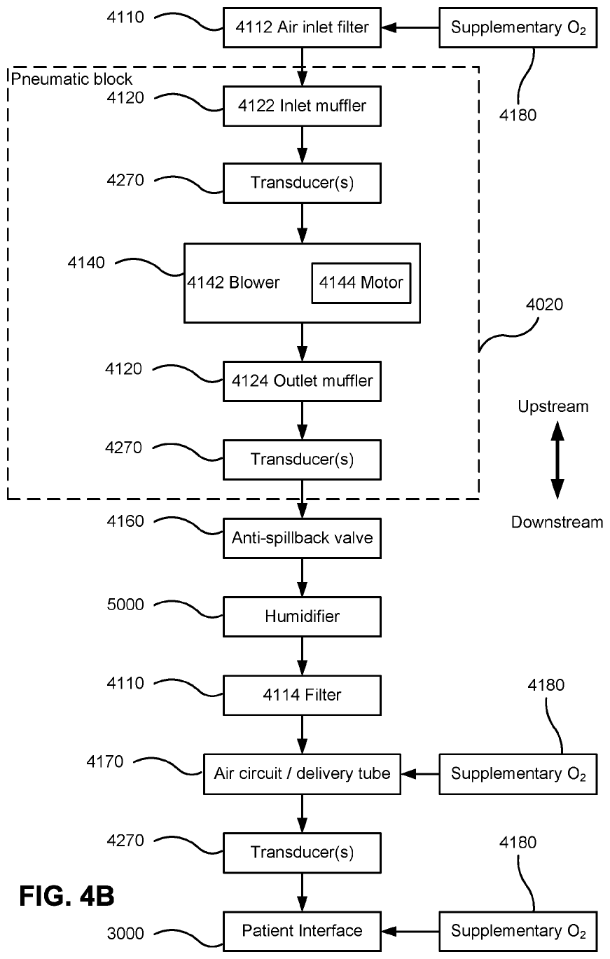

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

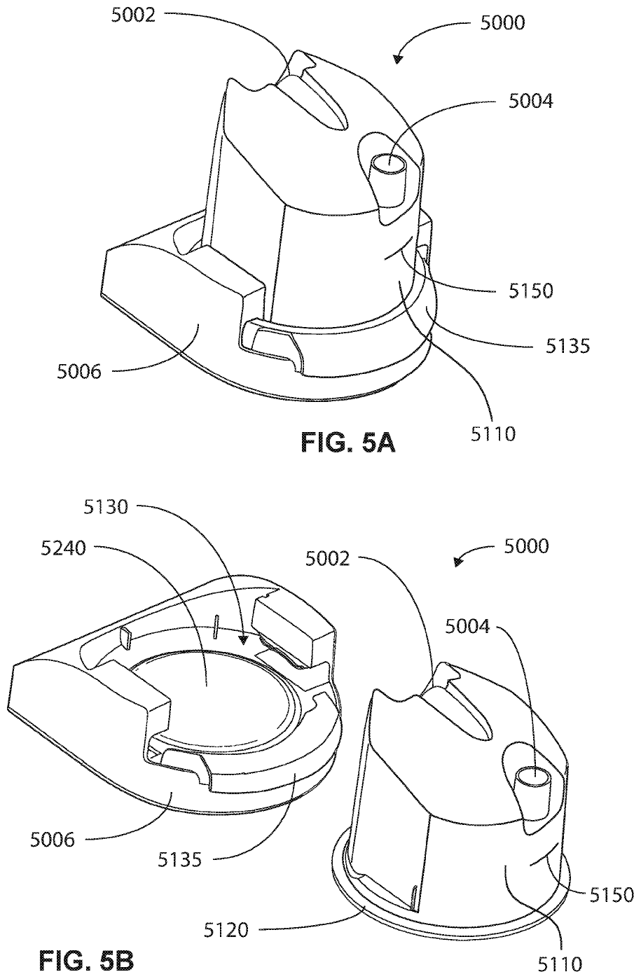

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Gas Flow Control Structure

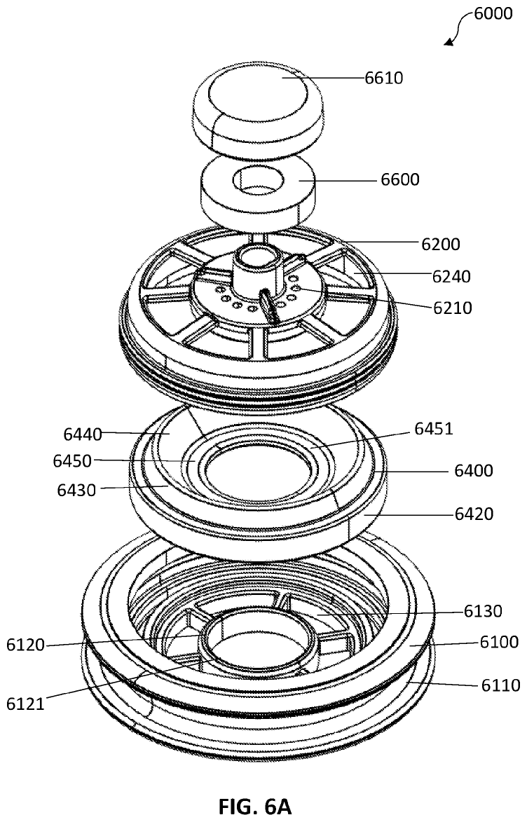

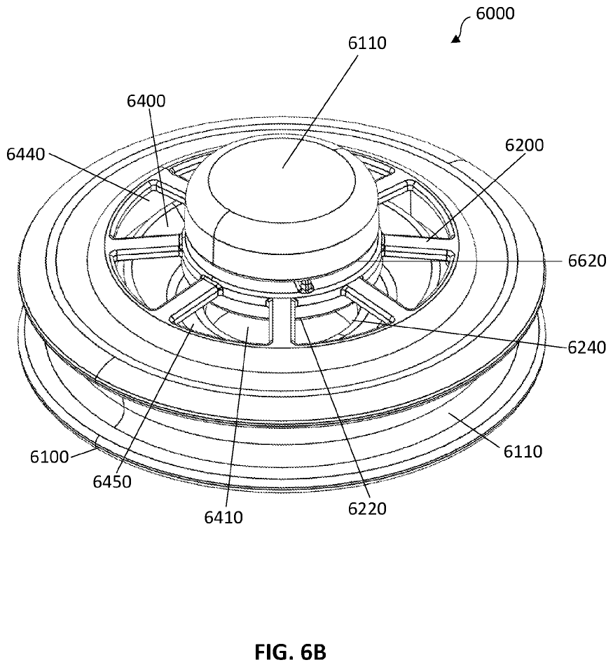

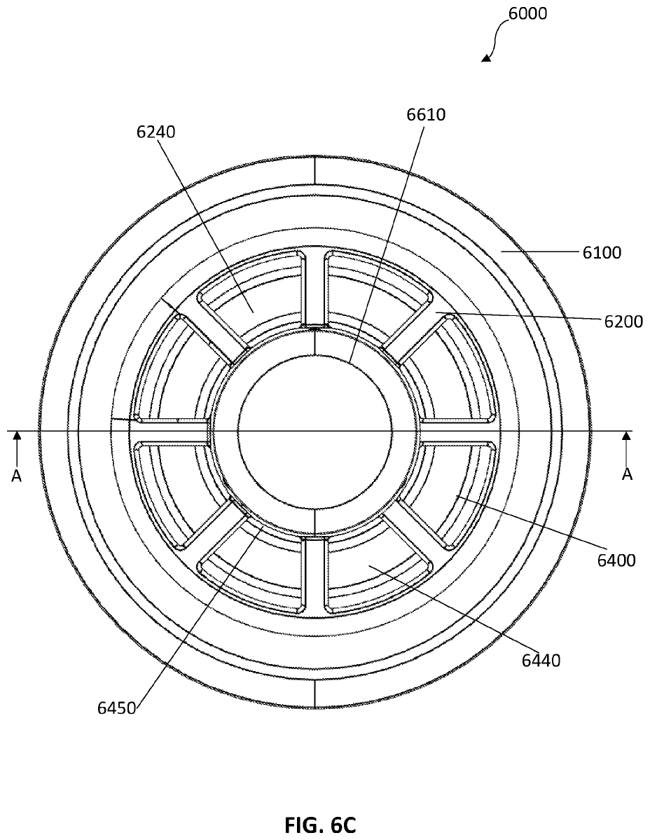

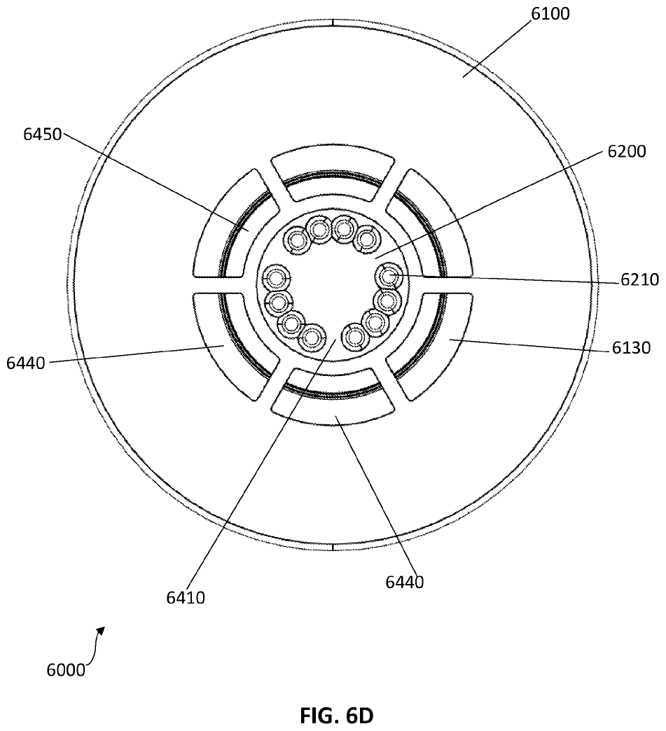

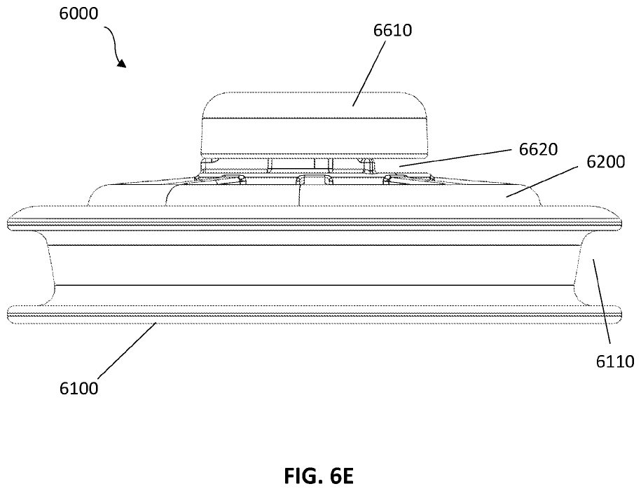

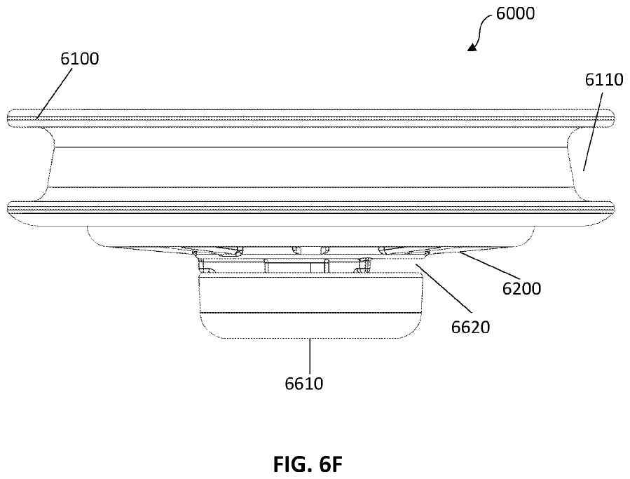

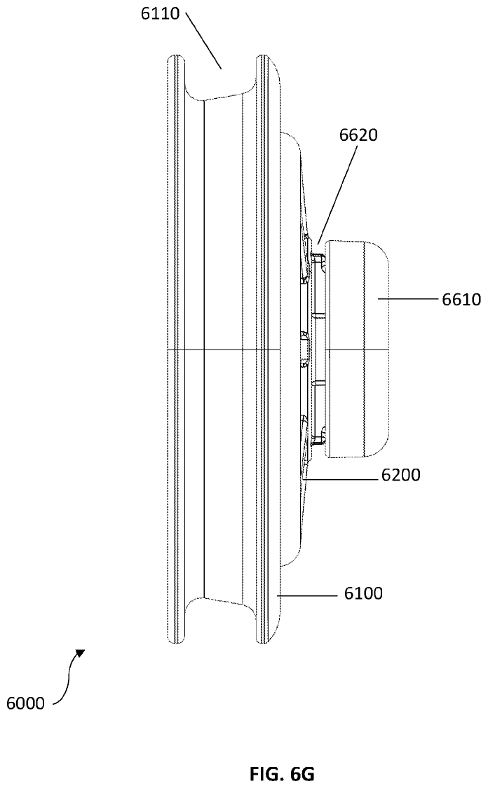

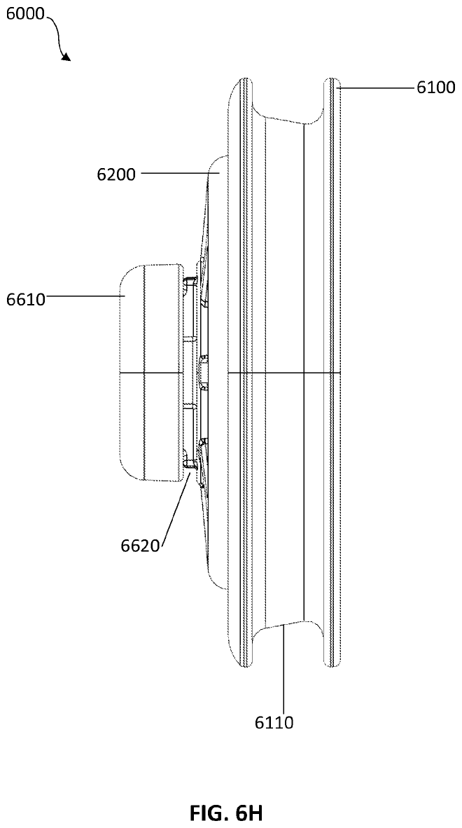

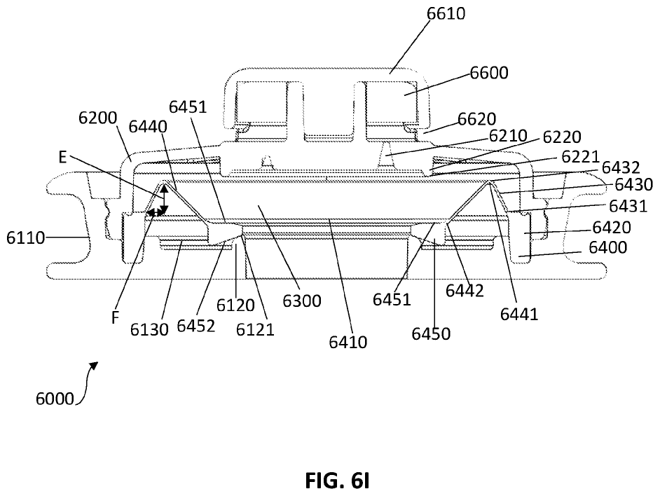

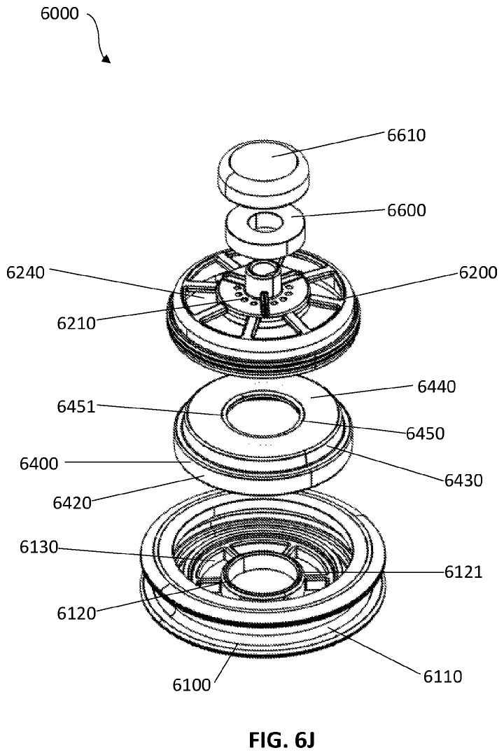

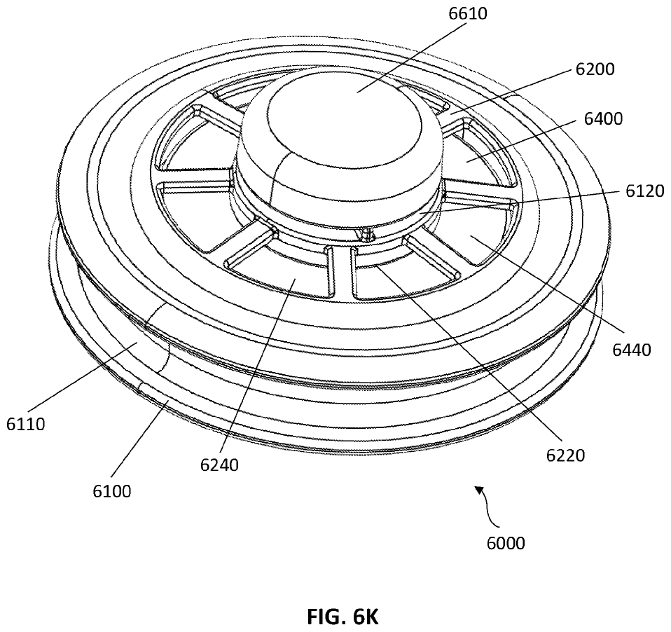

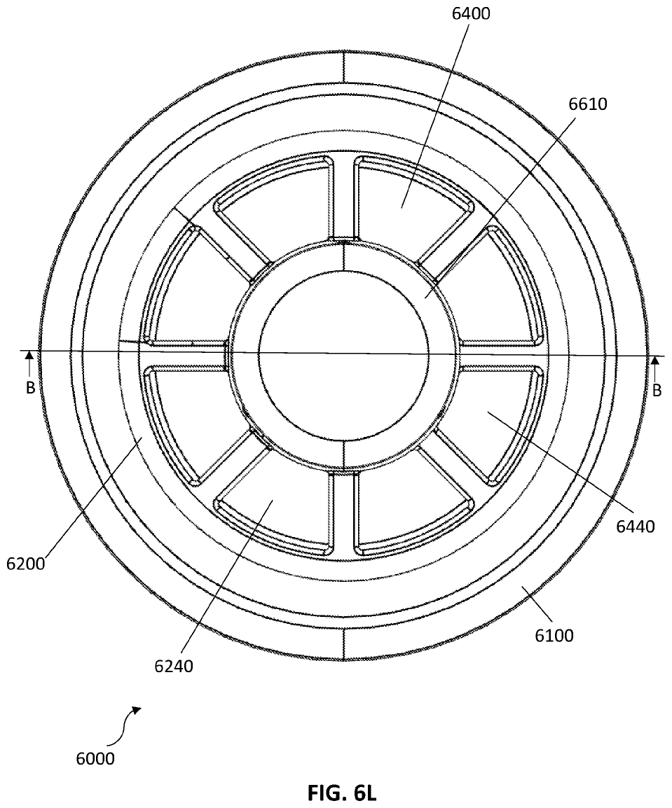

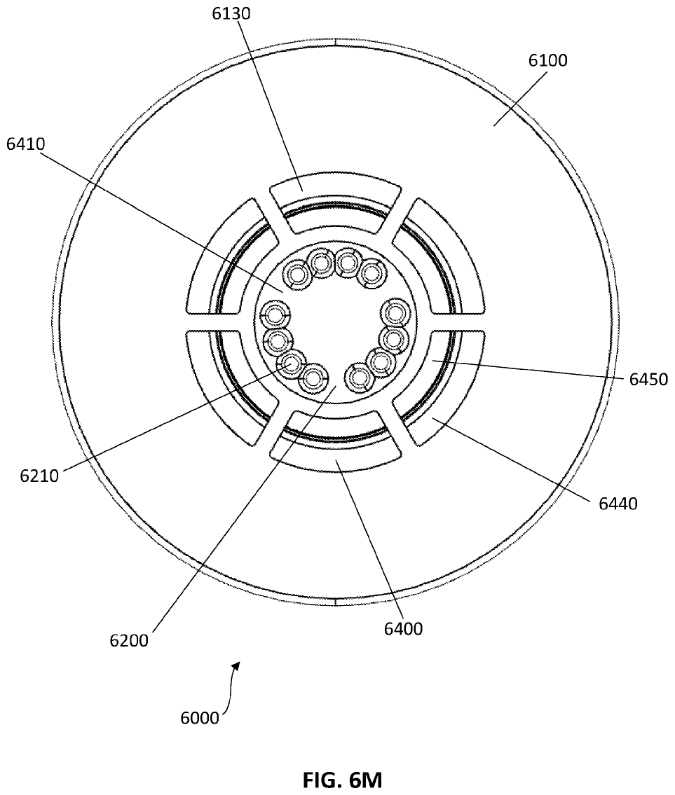

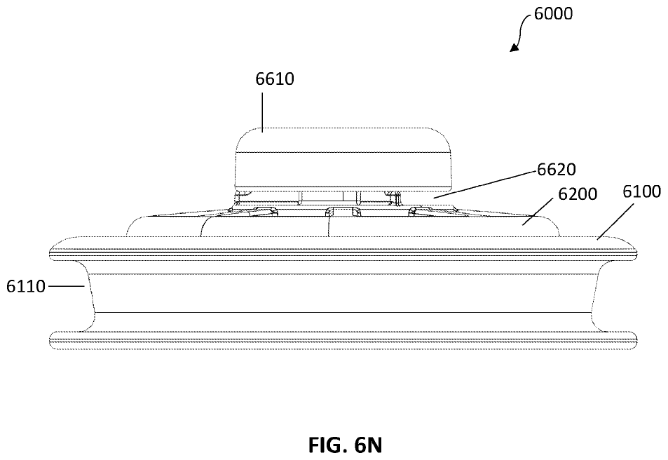

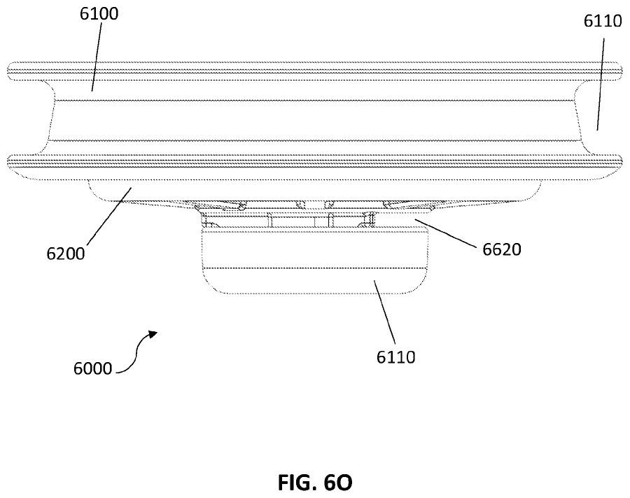

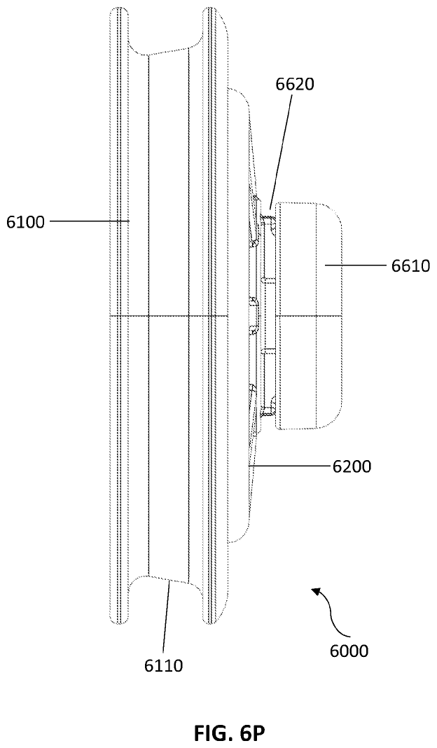

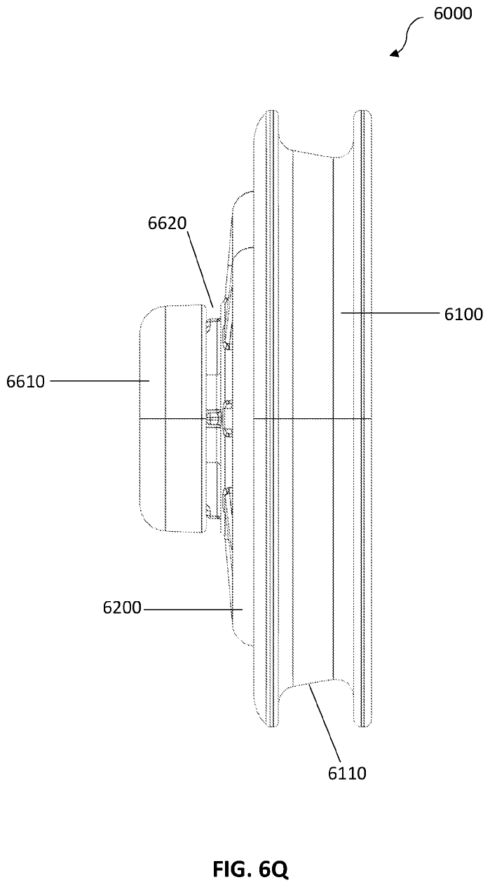

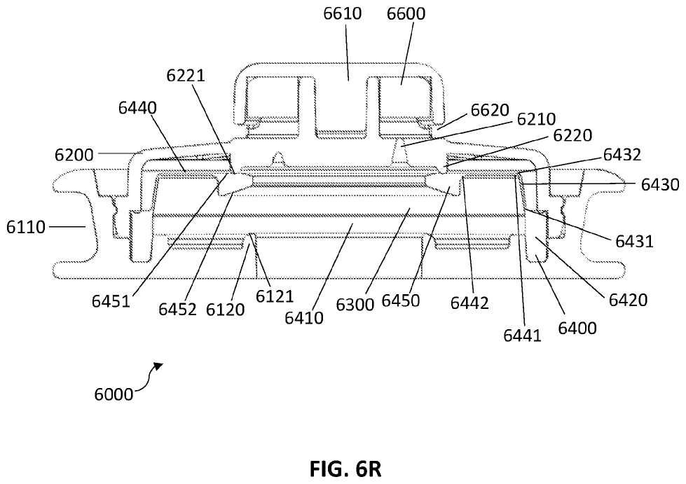

FIG. 6A shows an exploded view of a gas flow control structure according to one aspect of the present technology. The gas flow control structure is in an activated configuration.

FIG. 6B shows a perspective view of the gas flow control structure of FIG. 6A.

FIG. 6C shows a front view of the gas flow control structure of FIG. 6A.

FIG. 6D shows a back view of the gas flow control structure of FIG. 6A.

FIG. 6E shows a bottom view of the gas flow control structure of FIG. 6A.

FIG. 6F shows a top view of the gas flow control structure of FIG. 6A.

FIG. 6G shows a left side view of the gas flow control structure of FIG. 6A.

FIG. 6H shows a right side view of the gas flow control structure of FIG. 6A.

FIG. 6I shows a cross-sectional view along the plane A-A of FIG. 6C.

FIG. 6J shows an exploded view of the gas flow control structure of FIG. 6A in a deactivated configuration.

FIG. 6K shows a perspective view of the gas flow control structure of FIG. 6J.

FIG. 6L shows a front view of the gas flow control structure of FIG. 6J.

FIG. 6M shows a back view of the gas flow control structure of FIG. 6J.

FIG. 6N shows a bottom view of the gas flow control structure of FIG. 6J.

FIG. 6O shows a top view of the gas flow control structure of FIG. 6J.

FIG. 6P shows a left side view of the gas flow control structure of FIG. 6J.

FIG. 6Q shows a right side view of the gas flow control structure of FIG. 6J.

FIG. 6R shows a cross-sectional view along the plane B-B of FIG. 6L.

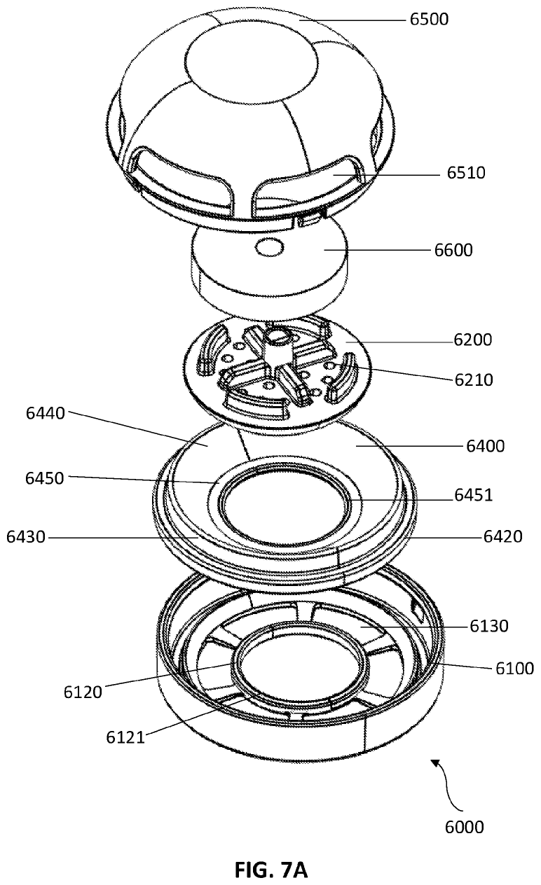

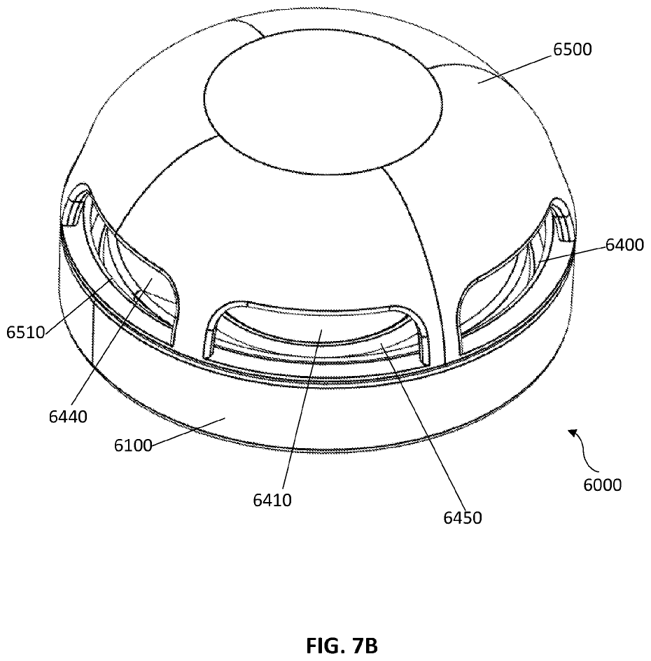

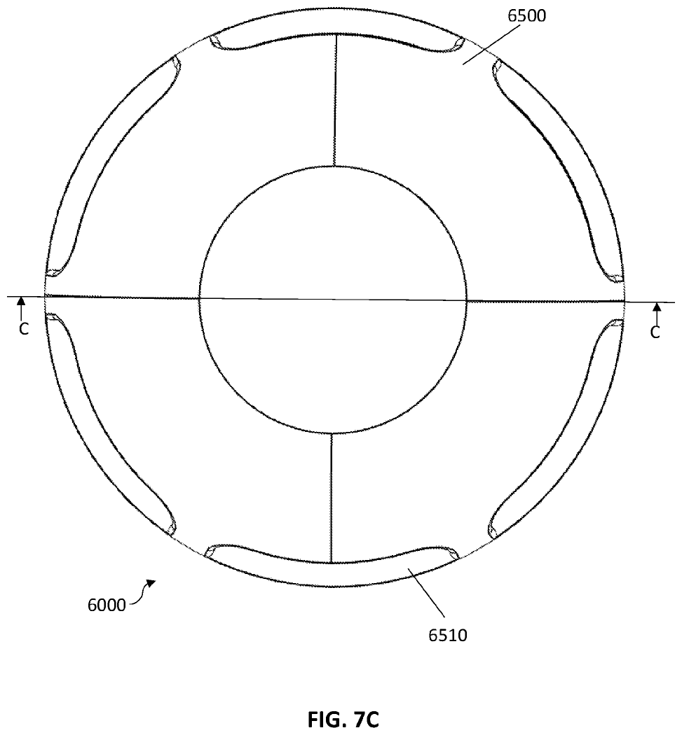

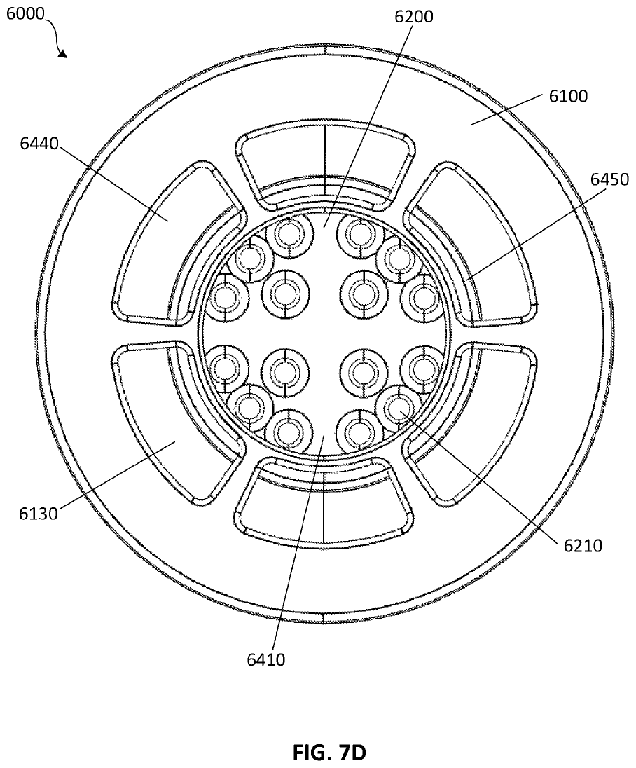

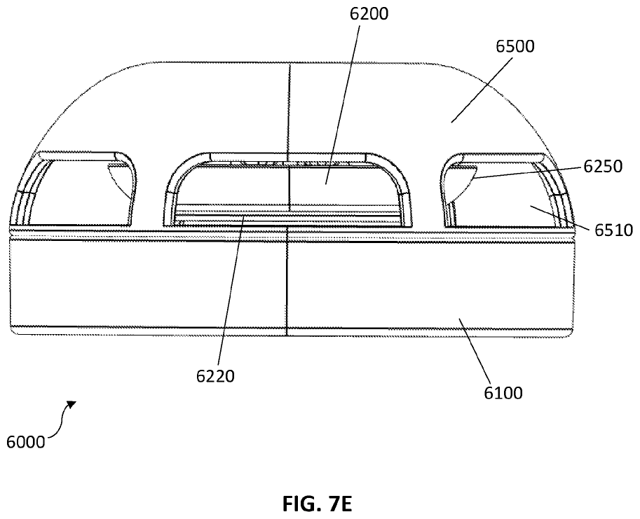

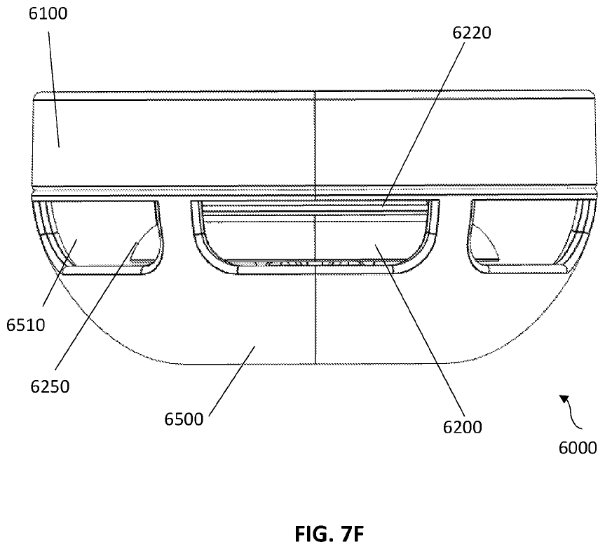

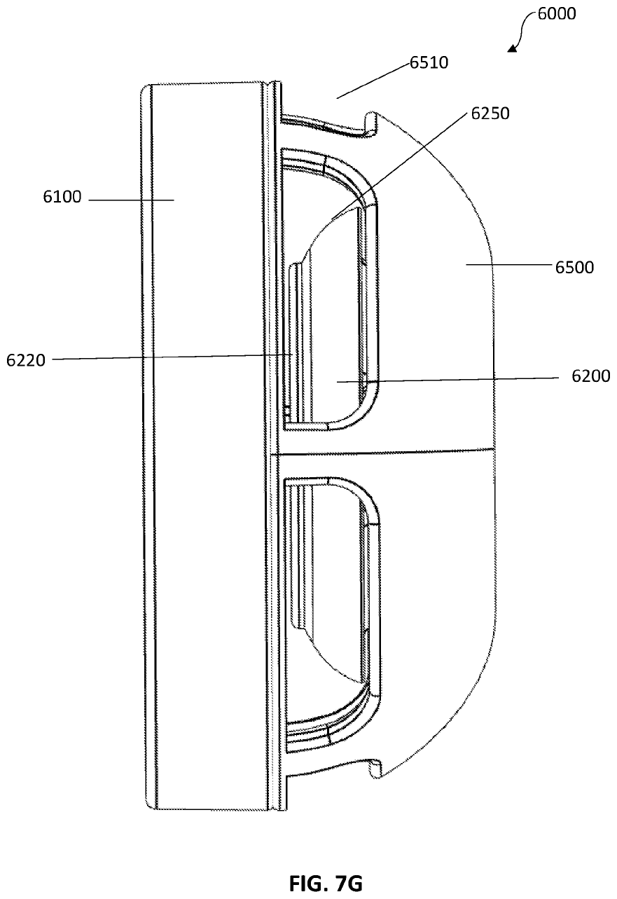

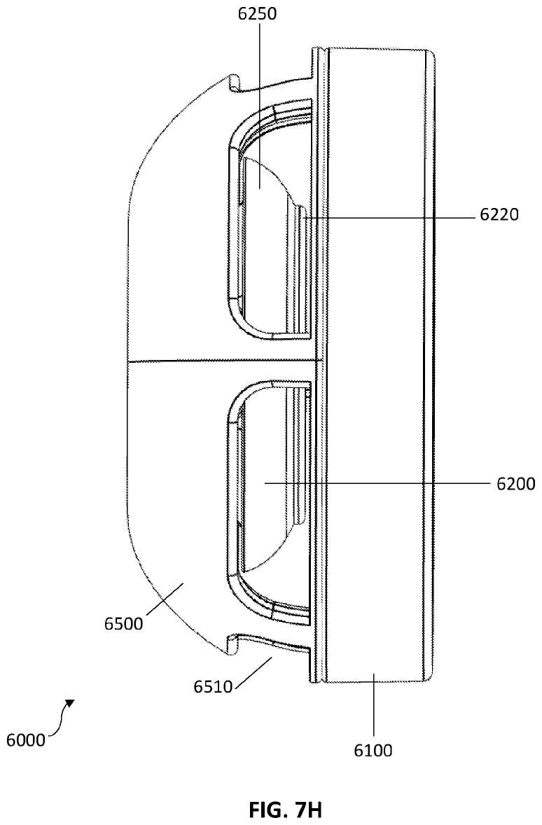

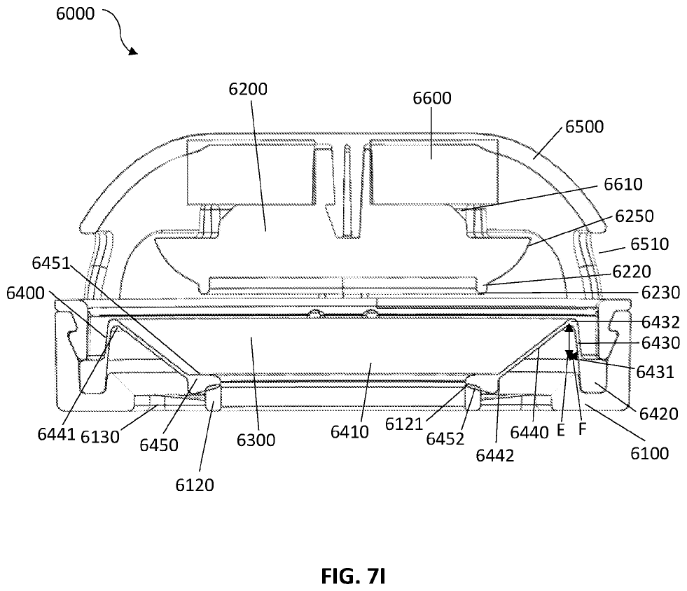

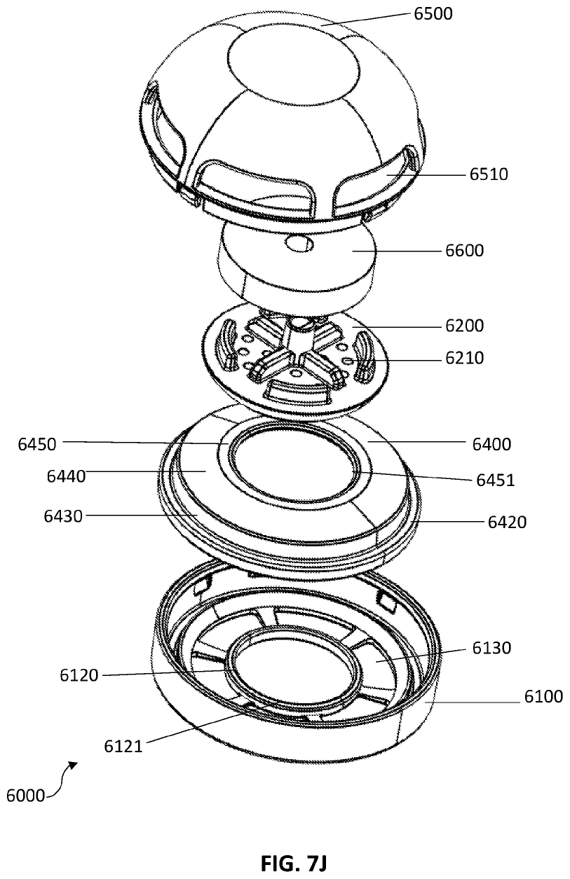

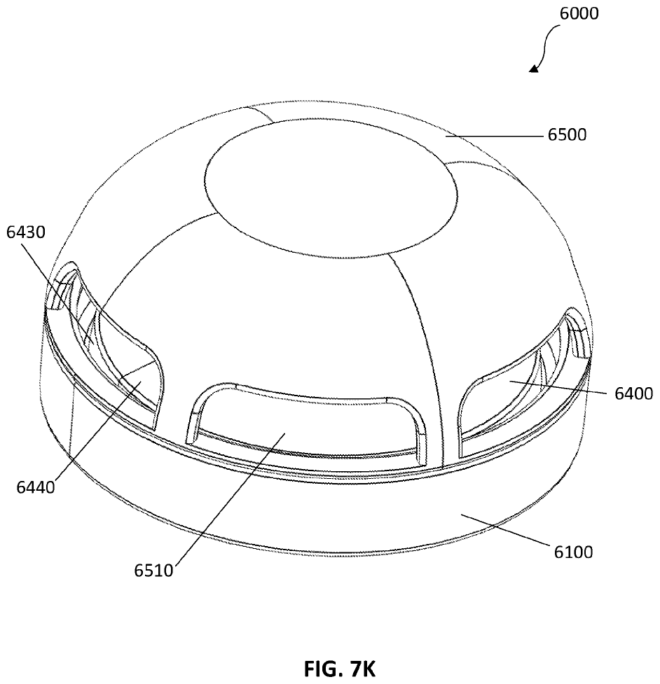

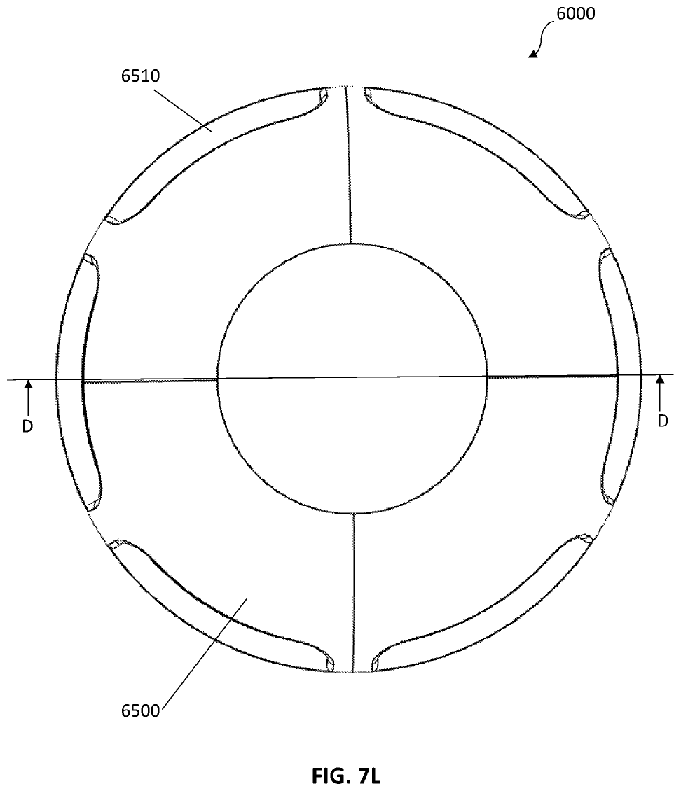

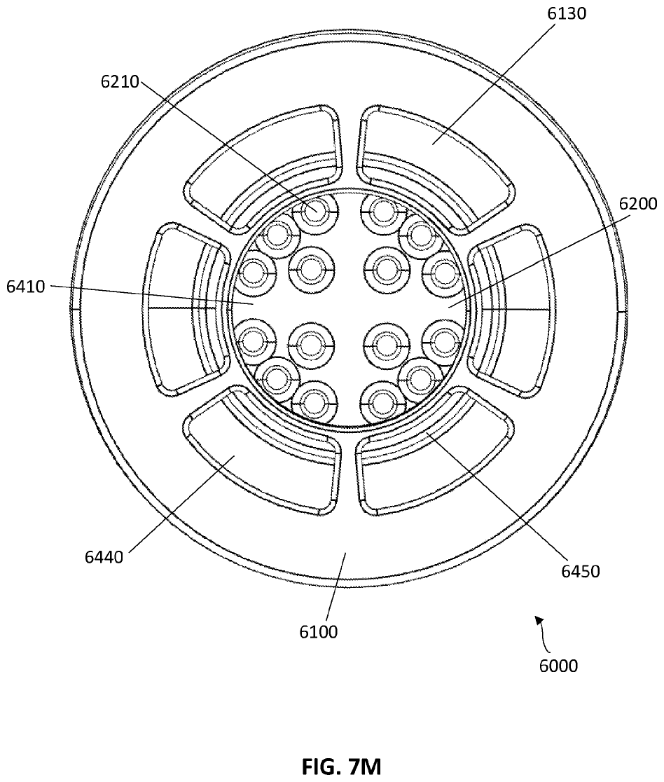

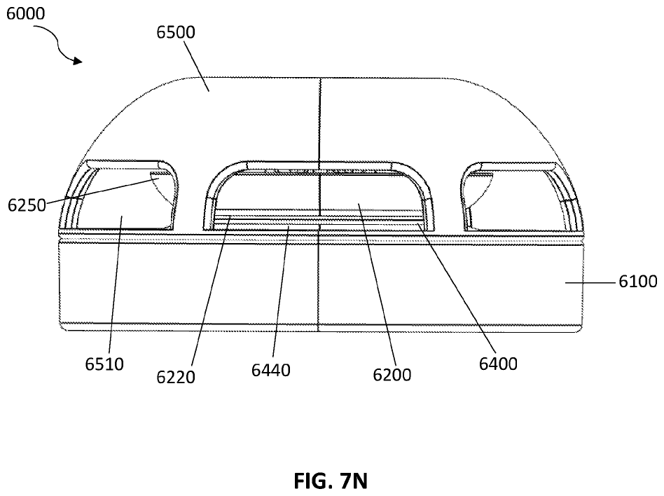

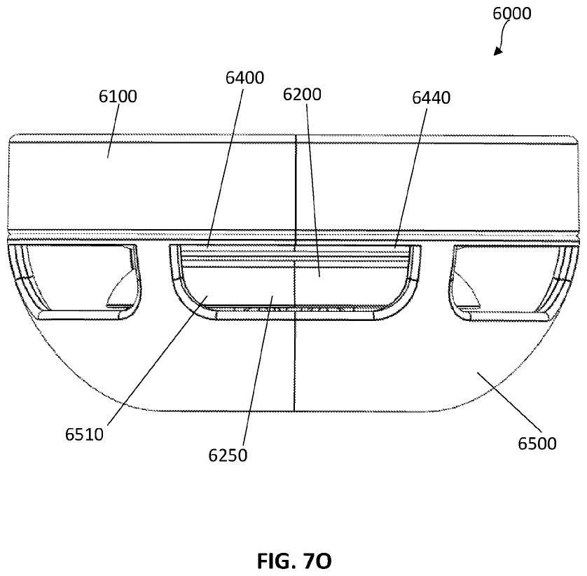

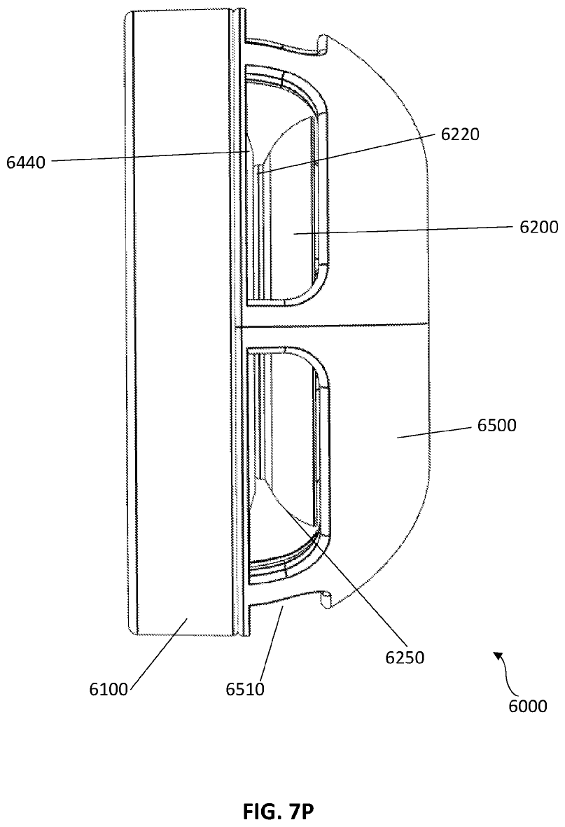

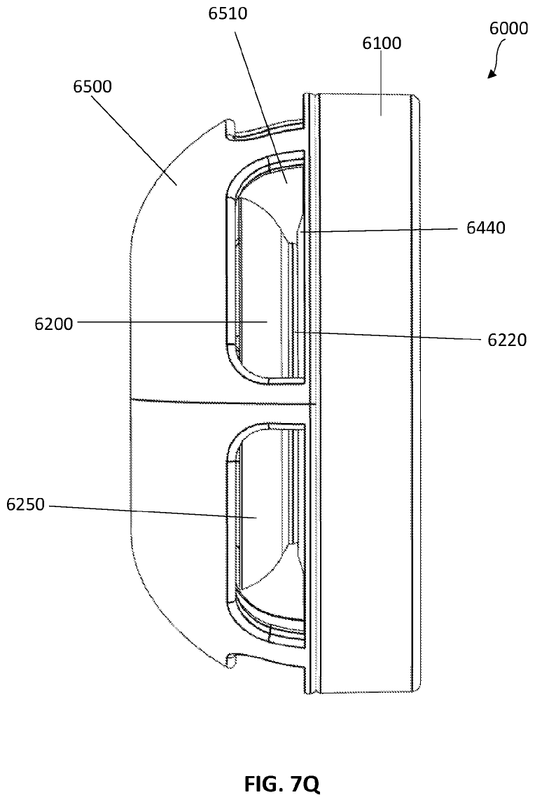

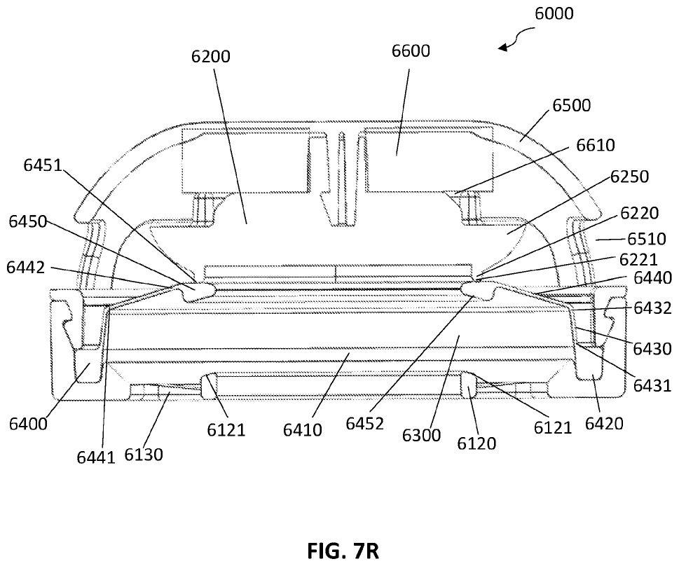

FIG. 7A shows an exploded view of a gas flow control structure according to another aspect of the present technology. The gas flow control structure is in an activated configuration.

FIG. 7B shows a perspective view of the gas flow control structure of FIG. 7A.

FIG. 7C shows a front view of the gas flow control structure of FIG. 7A.

FIG. 7D shows a back view of the gas flow control structure of FIG. 7A.

FIG. 7E shows a bottom view of the gas flow control structure of FIG. 7A.

FIG. 7F shows a top view of the gas flow control structure of FIG. 7A.

FIG. 7G shows a left side view of the gas flow control structure of FIG. 7A.

FIG. 7H shows a right side view of the gas flow control structure of FIG. 7A.

FIG. 7I shows a cross-sectional view along the plane C-C of FIG. 7C.

FIG. 7J shows an exploded view of a gas flow control structure of FIG. 7A in a deactivated configuration.

FIG. 7K shows a perspective view of the gas flow control structure of FIG. 7J.

FIG. 7L shows a front view of the gas flow control structure of FIG. 7J.

FIG. 7M shows a back view of the gas flow control structure of FIG. 7J.

FIG. 7N shows a bottom view of the gas flow control structure of FIG. 7J.

FIG. 7O shows a top view of the gas flow control structure of FIG. 7J.

FIG. 7P shows a left side view of the gas flow control structure of FIG. 7J.

FIG. 7Q shows a right side view of the gas flow control structure of FIG. 7J.

FIG. 7R shows a cross-sectional view along the plane D-D of FIG. 7L.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3B, a non-invasive patient interface 3000 in accordance with another aspect of the present technology comprises the following functional aspects: a seal-forming structure 3000, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 4.3.1 Seal-forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.1.7 Nasal Mask

In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice. This type of seal-forming structure 3100 may be referred to as a “nasal cushion” and a patient interface 3000 having such a seal-forming structure 3100 may be identified as a “nasal mask”.

### 4.3.1.8 Full Face Mask

In one form the patient interface 3000 comprises a seal-forming structure 3100 that forms a seal in use on a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “full face cushion” and the patient interface 3000 may be identified as a “full-face mask”.

### 4.3.1.9 Ultracompact Full-face Mask

In one form the patient interface 3000 comprises a seal-forming structure 3100 that forms a seal in use on a patient's chin region (which may include the patient's lip inferior and/or a region directly inferior to the lip inferior), to an inferior and or anterior surface of the patient's pronasale and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may be known as an ultra-compact full face mask and may comprise an ultra-compact full face cushion.

### 4.3.1.10 Nasal Cradle Mask

In one form, for example as shown in FIG. 3B, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of the patient's pronasale and to the patient's nasal alae. The seal-forming structure 3100 may seal to the patient's lip superior. This type of seal-forming structure 3100 may be referred to as a “cradle cushion”, “nasal cradle cushion” or “under-the-nose cushion”, for example.

The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

### 4.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 4.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another, suitable for a small sized head, but not a large sized head.

### 4.3.3.1 Conduit Headgear

### 4.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3B, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth). This allows the conduit of air circuit 4170 providing the flow of pressurised air to connect to a connection port 3600 of the patient interface in a position other than in front of the patient's face, for example on top of the patient's head.

Since air can be contained and passed through headgear tubing in order to deliver pressurised air from the air circuit 4170 to the patient's airways, the positioning and stabilising structure 3300 may be described as being inflatable. It will be understood that an inflatable positioning and stabilising structure 3300 does not require all components of the positioning and stabilising structure 3300 to be inflatable. For example, in the example shown in FIG. 3B, the positioning and stabilising structure 3300 comprises the tubes 3350, which are inflatable, and the strap 3310, which is not inflatable.

In the form of the present technology illustrated in FIG. 3B, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes is compressed to block or partially block the flow of gas along the tube, the other tube remains open to supply pressurised gas to the patient. In other examples of the technology, the patient interface 3000 may comprise a different number of tubes, for example one tube, or three or more tubes. In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head.

In the form of the technology shown in FIG. 3B the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used they may be indirectly connected together, for example each may be connected to a T-shaped connector having two arms/branches each fluidly connectable to a respective one of the tubes 3350 and a third arm or opening providing the connection port 3600 for fluid connection to the air circuit 4170 in use.

The tubes 3350 may be formed from a flexible material, such as an elastomer, e.g. silicone or TPE, or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

As described in U.S. Pat. No. 6,044,844, the contents of which are incorporated herein, the tubes 3350 may be crush resistant to avoid the flow of breathable gas through the tubes being blocked if either is crushed during use, for example if it is squashed between a patient's head and pillow. Crush resistant tubes may not be necessary in all cases as the pressurised gas in the tubes may act as a splint to prevent or at least restrict crushing of the tubes 3350 during use. A crush resistant tube may be advantageous where only a single tube 3350 is present as if the single tube becomes blocked during use the flow of gas would be restricted and therapy will stop or reduce in efficacy. In some examples, the tubes 3350 may be sized such that each tube 3350 is able to provide sufficient flow of gas to the plenum chamber 3200 on its own should one of the tubes 3350 become blocked.

Each tube 3350 may be configured to receive a flow of air from the connection port 3600 on top of the patient's head and to deliver the flow of air to the seal-forming structure 3100 at the entrance of the patient's airways. In the example shown in FIG. 3B, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force vector in a posterior and superior direction (e.g. a posterosuperior direction). A sealing force vector with a posterosuperior direction may facilitate the seal-forming structure 3100 forming a good seal to both the inferior periphery of the patient's nose and the anterior-facing surfaces of the patient's face on either side of the patient's nose and the patient's lip superior.

### 4.3.3.1.2 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3B, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 4.3.3.1.3 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or like sound which may be easy for a patient to use as the patient may know for sure that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 4.3.3.1.4 Conduit Headgear Straps

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3B, the patient interface 3000 comprises a strap 3310 forming part of the positioning and stabilising structure 3300. The strap 3310 may be known as a back strap or a rear headgear strap, for example. In other examples of the present technology, one or more further straps may be provided. For example, patient interfaces 3000 according to examples of the present technology having a full face cushion may have a second, lower, strap configured to lie against the patient's head proximate the patient's neck and/or against posterior surfaces of the patient's neck.

In the example shown in FIG. 3B, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 3B, the positioning and stabilising structure 3300 comprises a pair of tabs 3320. In use a strap 3310 may be connected between the tabs 3320, The strap 3310 may be sufficiently flexible to pass around the back of the patient's head and lie comfortably against the patient's head, even when under tension in use.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel. Decoupling structures according to certain forms of the technology are described further below. A further alternative, is locating the vent 3400 in a part of the conduit headgear, for example on tubes 3350.

The vent 3400 may also be part of a gas flow control structure 6000. Gas flow control structures 6000 according to certain forms of the technology are described in detail later in this specification.

### 4.3.5 Decoupling Structure(s)

In one form the patient interface 3000 includes at least one decoupling structure, for example, a swivel or a ball and socket. In some forms, the decoupling structure may be part of an elbow structure or connection member located between, and configured to fluidly connect, the plenum chamber 3200 and the air circuit 4170.

### 4.3.6 Connection Port

Connection port 3600 allows for connection to the air circuit 4170.

### 4.3.7 Forehead Support

In one form, the patient interface 3000 includes a forehead support 3700.

### 4.3.8 Anti-asphyxia Valve

In one form, the patient interface 3000 includes an anti-asphyxia valve (AAV). In other forms, the AAV may be located in an elbow structure or connection member located between the plenum chamber 3200 and the air circuit 4170. Alternatively, the AAV may be located in a part of the conduit headgear, for example tubes 3350. An AAV may alternatively be referred to as a non-rebreathing valve (NRV).

The anti-asphyxia valve may also be part of a gas flow control structure 6000. Gas flow control structures 6000 according to certain forms of the technology are described in detail later in this specification.

### 4.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

In some forms of the present technology, an oxygen diverter valve (ODV) is provided to the respiratory system to prevent the supplementary oxygen supply flowing into the RPT device 4000 when there is no supply of pressurised air from the RPT device 4000, for example if there is a fault in the RPT device.

### 4.4 Gas Flow Control Structure

### 4.4.1 Gas Flow Control Structure Overview

As mentioned earlier a respiratory therapy system according to certain forms of the technology may comprise a vent 3400 to allow for the washout of exhaled gases, e.g. carbon dioxide.

Also, as mentioned earlier a respiratory therapy system according to certain forms of the technology may include an anti-asphyxia valve (AAV) that opens to provide a flow path between the patient's airways and the ambient atmosphere in the event of a cessation of the supply of breathable gas from the RPT device 4000, or a drop in pressure of the supply of breathable gas from the RPT device 4000 to below a certain pressure level. Thus, the AAV reduces the risk of excessive carbon dioxide rebreathing by a patient in such circumstances.

In certain aspects of the present technology, a gas flow control structure 6000 is provided that is configured to act as both a vent and an AAV in use. FIGS. 6A to 6R show a gas flow control structure 6000 according to one form of the technology and FIGS. 7A to 7R show a gas flow control structure 6000 according to another form of the technology.

In the illustrated forms, the gas flow control structure 6000 is located in the respiratory therapy system during use such that it separates a first volume that is pressurised during use and a second volume that is the surrounding ambient air. The gas flow control structure 6000 also controls the flow of gas between the first volume and the second volume. The first volume may be a volume inside any one or more of the components of the respiratory therapy system and be pressurised by the flow of gas generated by the RPT device 4000. For example, the first volume may be a volume inside any one or more of: plenum chamber 3200; air circuit 4170; conduit headgear (for example tubes 3350); elbow structure; or connection member.

In the illustrated forms, the gas flow control structure 6000 comprises a base member 6100. The gas flow control structure 6000 further comprises a vent member 6200 comprising at least one vent opening 6210 through which a flow of gas can pass from the first volume to the second volume during use.

In the illustrated forms, the gas flow control structure 6000 further comprises an AAV opening 6300 and AAV member 6400. The AAV opening 6300 is formed between the base member 6100 and the vent member 6200. The AAV member 6400 is mounted to the base member 6100 and is movable between an activated configuration, shown in FIGS. 6A-6I and 7A-7I, in which gas can flow between the first volume and the second volume through the AAV opening and a deactivated configuration, shown in FIGS. 6J-6R and 7J-7R, in which the AAV member 6400 forms a seal with a portion of the vent member 6200 so gas is blocked from flowing through the AAV opening 6300.

In some forms, the base member 6100 is generally located on a side of the gas flow control structure 6000 adjacent the first volume and the vent member 6200 is located on a side of the gas flow control structure 6000 adjacent the second volume with the AAV member 6400 located between the base member 6100 and the vent member 6200. The base member 6100 may also comprise surfaces that are exposed to the second volume during use.

In forms of the technology the size of the AAV opening 6300 is preferably large enough to allow for effective carbon dioxide washout when there is no supply of pressurised air to the patient interface 3000. If the size of the AAV opening 6300 is too small, the flow resistance will be too high and carbon dioxide does not wash-out effectively and instead flows into the air circuit 4170. Generally, the smaller the air circuit 4170, the less sensitive to the size of the AAV opening 6300 the rate of carbon dioxide washout is since the AAV opening 6300 is less likely to be the highest point of flow resistance. In forms of the technology the size of the vent opening 6210 is also preferably large enough to allow for effective carbon dioxide washout when there is a supply of pressurized air to the patient interface 3000.

The AAV member 6400 is biased towards the activated configuration in which gas can flow between the first volume and the second volume through the AAV opening 6300. When the first volume contains gas having a pressure that is below an activation pressure, the AAV member 6400 is in the activated configuration. That is, the activation pressure is the pressure of the gas in the first volume, below which the force of the pressure acting on the AAV member 6400 is insufficient to overcome the bias and the bias urges the AAV member 6400 into the activated configuration. In the form of the technology shown in FIGS. 6A-7R, in the activated configuration a portion of the AAV member 6400 may contact a portion of the base member 6100. In other aspects, in the activated configuration the AAV member 6400 does not contact the base member 6100 or any part of the gas flow control structure 6000.

When the first volume contains gas having a pressure that is above a deactivation pressure, the AAV member 6400 is in the deactivated configuration and forms a seal with a portion of the vent member 6200 so gas is blocked from flowing through the AAV opening 6300. That is, the deactivation pressure is the pressure of the gas in the first volume, above which the force of the pressure acting on the AAV member 6400 is sufficient to overcome the bias and to urge the AAV member 6400 into the deactivated configuration.

In some forms, the activation pressure and the deactivation pressure may be substantially the same. In other forms, the activation pressure and the deactivation pressure may be different. The activation pressure and the deactivation pressures may be below the pressure of the supply of the flow of gas. In some forms the activation pressure and the deactivation pressures are below 4 cmH₂O, and in some forms they may be below 3 cmH₂O.

During use, the gas flow control structure 6000 is configured to allow a flow of gas through the at least one vent opening 6210 in the vent member 6200 from the first volume to the second volume in both the activated and deactivated configurations. When the RPT device 4000 is functioning normally and a supply of gas at positive pressure is being supplied to the patient 1000, where the pressure in the first volume is greater than the deactivation pressure, the AAV member 6400 moves to the deactivated configuration in which the AAV opening 6300 is blocked. The force of the pressurised gas in the first volume acts on the AAV membrane 6400 to move it to the deactivated configuration. In some forms of the technology the deactivation pressure is between approximately 1.5 and 2.5 cmH₂O.

When the RPT device 4000 is malfunctioning or for some other reason the pressure in the first volume falls below the activation pressure, the AAV member 6400 moves to the activated configuration in which the AAV opening 6300 is open as a result of the bias. This allows gas to flow between the first volume and second volume so that the patient is not rebreathing carbon dioxide. In some forms of the technology the activation pressure is between approximately 0.5 and 1.5 cmH₂O.

When the pressure in the first volume is between the deactivation pressure and the activation pressure, the AAV member 6400 will remain in whichever configuration it is currently in. For instance, if the pressure rises above the activation pressure but doesn't reach the deactivation pressure, the AAV member 6400 will stay in the activated configuration and if the pressure falls below the deactivation pressure but doesn't reach the activation pressure, the AAV member 6400 will stay in the deactivated configuration.

### 4.4.2 AAV Member

In the illustrated forms of the technology, the AAV member 6400 is a flexible, resilient membrane which is substantially annular in shape when projected on to a plane. The AAV member 6400 is arranged as part of the gas flow control structure 6000 such that, in use, gas from the first volume flows through the central hole 6410 formed by the annular shape of the AAV member 6400 when passing to the second volume through the at least one vent opening 6210. In some forms, the AAV member 6400 may have an annular shape with a circular outer rim and a circular central hole 6410. In other forms the AAV member 6400 may have an oval-shape, for instance with an ovular outer rim and/or ovular central hole 6410. The AAV member may be formed from silicone, rubber or another elastic material.

As described above, in certain forms, the AAV member 6400 is able to move between an activated configuration and a deactivated configuration. Exemplary forms of an AAV member 6400 and the shapes adopted in each of these configurations are described in more detail below. The AAV member 6400 may be configured so that it is biased towards the activated configuration. That is, the AAV member 6400 has an elastic property that causes it to resiliently return to the activated configuration if urged into the deactivated configuration. This resilience may result from the material from which the AAV member 6400 is formed and/or its configuration. For example, the AAV member 6400 may be formed (for example by molding) in a ‘neutral’ configuration (i.e. the configuration adopted by the AAV member 6400 when no forces are applied to it) from an elastic material such as silicone or rubber. Additionally, or alternatively, the AAV member 6400 may be formed with angled segments having a natural angle to each other (as described further in the examples below) so that a change in angles causes the segments to resiliently return to the natural angle. Consequently, when the AAV member 6400 is deformed into another configuration (such as the deactivated configuration, as described below), the AAV member 6400 has an inherent bias to return to the neutral configuration. In some forms, the activated configuration of the AAV member 6400 may be the same as the neutral configuration. In other forms, the activated configuration of the AAV member 6400 may be similar to the neutral configuration.

The AAV member 6400 may comprise a mounted part 6420 that is mounted to the base member 6100. The mounted part 6420 may be friction fit, glued, or fixedly connected by some other means to the base member 6100. The mounted part 6420 may be formed around the entire outer circumference of the annular shape of the AAV member 6400. Alternatively, there may be a plurality of mounted parts 6420 located around the outer circumference that mount the AAV member 6400 to the base member 6100. The mounted part 6420 may be formed as a thicker portion of the AAV member 6400 and as shown in FIGS. 6I, 6R, 7I, and 7R may have a substantially rectangular cross-sectional shape.

The AAV member 6400 may comprise a first segment 6430 with a first end 6431 and an opposite second end 6432. The first end 6431 of the first segment 6430 is rotatably connected to the mounted part 6420 of the AAV member at a first rotatable connection.

The AAV member 6400 may comprise a second segment 6440 with a first end 6441 and an opposite second end 6442. The first end 6441 of the second segment 6440 is rotatably connected to the second end 6432 of the first segment 6430 at a second rotatable connection.

The AAV member 6400 may further comprise a foot portion 6450 that is rotatably connected to the second end 6442 of the second segment 6440 at a third rotatable connection. The foot portion 6450 may be the portion of the AAV member 6400 that contacts the portion of the vent member 6200 in the deactivated configuration. As shown in FIGS. 6R and 7R a substantially flat surface 6451 of the foot portion 6450 may contact the portion of the vent member 6200 in the deactivated configuration, where the foot portion 6450 is configured so that the flat surface 6451 is oriented substantially perpendicular to the central axis 6410 in the deactivated configuration. In the activated configuration, the contact between the foot portion 6450 and the vent member 6200 may form a seal such that no gas flows through the AAV opening 6300.

In some forms, as shown in FIGS. 6A to 7R, the first 6430 and second 6440 segments, the mounted part 6420 and the foot portion 6450 are all annular in shape and have the same central axis as the central hole 6410 of the AAV member 6400. The first 6430 and second 6440 segments, the mounted part 6420 and the foot portion 6450 may be arranged concentrically with the mounted part 6420 furthest from the central axis of the central hole 6410 followed by the first segment 6430, then the second segment 6440 and with the foot portion 6450 closest to the central axis 6410. In some forms, the central axis of the central hole 6410 is coincident with an axis of rotational symmetry of the AAV member 6400 such that the cross-sectional shape of the AAV member 6400 is the same along any plane on which the central axis 6410 lies.

The first segment 6430 and second segment 6440 may be formed as thin membrane-like sections of the AAV member 6400. In some forms, the first 6430 and second 6440 segments are substantially planar. The first 6430 and second 6440 segments may be resilient such that, if placed under pressure from gas in the first volume, they are able to distort without breaking and will return to their original shape and configuration when no longer under pressure.

The foot portion 6450 may also be the portion of the AAV member 6400 that contacts the base member 6100 when in the activated configuration. As shown in FIGS. 6I and 7I an angled surface 6452 of the foot portion 6450 may contact the portion of the base member 6100 in the activated configuration, where the angled surface 6452 may be a plane surface at an angle not perpendicular to the central axis of the central opening 6421 when the AAV member 6400 is in the activated and/or deactivated configurations. The angled surface 6452 of the foot portion 6450 may be on an opposite side of the foot portion 6450 to the flat surface 6451. In some forms, the gas flow control structure 6000 is configured so that the foot portion 6450 forms a seal with the base member 6100 when in the activated configuration. In other forms, the gas flow control structure 6000 is configured so that the foot portion 6450 does not contact the base member 6100 when in the activated configuration.

The angled surface 6452 may be on a side of the foot portion 6450 closest to, and facing, the base member 6100 and therefore on a side of the foot portion 6450 exposed to the first volume when the AAV member 6400 is in the deactivated configuration. The flat surface 6451 may be on a side of the foot portion closest to the vent member 6200 and therefore on a side exposed to the second volume when the AAV member 6400 is in the deactivated configuration. In some forms, the foot portion 6450 may be formed as a thicker portion of the AAV member 6400 compared to the first 6430 and second 6440 segments, and as shown in FIGS. 6I, 6R, 7I, and 7R, may have a substantially trapezoidal cross-sectional shape. In other forms, the cross-sectional shape of the mounted part 6420 and the foot portion 6450 may be another shape, for instance square, triangular, circular, or some other polygon. In some forms, the cross-sectional shape of the foot portion 6450 has curved corners. The shape of the foot portion 6450 may assist in achieving a suitable seal between the foot portion 6450 and the vent member 6200 in the deactivated configuration and/or suitable contact between the foot portion 6450 and the base member 6100 in the activated configuration.

In some forms, as shown in FIGS. 6I, 6R, 7I, and 7R, the foot portion 6450 may only change position along the central axis of the central hole 6410 between the activated and deactivated configuration. There may be no angular change in position of the foot portion 6450 with respect to the base member 6100 and/or vent member 6200.

In some forms, portions of the first segment 6430 and second segment 6440 may balloon out in a direction away from the first volume when in the deactivated configuration as a result of pressurised gas in the first volume acting on the segments. In some forms, it may be the portions of the first segment 6430 and second segment 6440 adjacent the second rotatable connection that balloon out. The ballooning out may result in the direct distance between the first end 6431 and second end 6432 of the first segment 6430 decreasing and/or the distance between the first end 6441 and second end 6442 of the second segment 6440 decreasing. The decreasing distance may result in the foot portion 6450 moving outwards to a position further from the central longitudinal axis of the central hole 6410. The flat surface 6451 of the foot portion 6450 may be configured with a large enough area such that a seal can be achieved between the foot portion 6450 and the vent member 6200 when the foot portion 6450 is in a variety of positions, including when the foot portion 6450 moves outwards to a position further from the central longitudinal axis of the central hole 6410 as a result of the described ballooning. That is, the dimensions of the foot portion 6450 enable a seal to be achieved between the foot portion 6450 and the vent member 6200 with some tolerance to deformation of the AAV member 6400 resulting from the action of pressurised gas on the AAV member 6400.

In other forms, the second segment 6440 may be the portion of the AAV member 6400 that forms a seal with the portion of the vent member 6200 in the deactivated configuration. The second segment 6440 may also be the portion of the AAV member 6400 that contacts the base member 6100 when in the activated configuration, e.g. a region of the second segment 6440 closest to the central axis 6410 forms a seal with the portion of the vent member 6200 and/or contacts the base member 6100. That is, in some forms, there may be no foot portion 6450 as part of the AAV member 6400 and the function of the foot portion 6450 is achieved by the second segment 6440.

In some forms, the AAV member 6400 is integrally formed in one piece. The first, second, and third rotatable connections may be parts of the AAV member 6400 that are flexible, for instance they may be formed by bends or curves in the AAV member 6400 that allow for greater flexibility than other parts of the AAV member 6400. In some forms, the AAV member 6400 may have a smaller thickness at the first, second, and third rotatable connections compared to other parts of the AAV member 6400 to allow for greater flexibility at these points compared to other parts of the AAV member 6400. In other aspects, the rotatable connections may be formed by joins between the different parts of the AAV member 6400, the joins being flexible such that the adjacent parts of the AAV member 6400 can rotate around the joins. An advantage of the one-piece construction for the AAV member 6400 is that the joins between segments are inherently sealed.

In some forms, as shown in FIGS. 6I and 7I, the first segment 6430 and the second segment 6440 of the AAV membrane 6400 may be oriented relative to each other to form a V-shape in cross-section when in the activated configuration. In the activated configuration the angle between the first segment 6430 and the second segment 6440 at the second rotatable connection may be between 45° and 80°, in some aspects the angle is between 60° and 65°. In some forms, as shown in FIGS. 6R and 7R, the first segment 6430 and the second segment 6440 of the AAV membrane 6400 may be oriented relative to each other to form a V-shape in cross-section when in the deactivated configuration. The V-shape in the deactivated configuration may have a greater angle than in the activated configuration. In some examples the angle of the V-shape may be obtuse in the deactivated configuration and acute in the activated configuration. As shown in FIGS. 6R and 7R, in the deactivated configuration the angle between the first segment 6430 and the second segment 6440 at the second rotatable connection is greater than 90°, in some aspects the angle is between 90 and 110°. The angle between the first segment 6430 and the second segment 6440 may be altered by changing other parameters of the AAV member 6400 as discussed below, such as the distance between the second rotatable connection and the mounted part 6420 along both the parallel and perpendicular directions to the central axis of the central hole 6410. The angle between the first segment 6430 and the second segment 6440 may also be dependent on the lengths of the first segment 6430 and the second segment 6440 and the distance between the mounted part 6420 and the central hole 6410.

In some forms, as shown in FIGS. 6I, 6R, 7I, and 7R, the second segment 6440 may not completely flip or invert between the activated and the deactivated configurations. The angle between the second segment 6440 and a plane perpendicular to the central axis of the central hole 6410 may be larger in the activated configuration compared to the deactivated configuration. In the deactivated configuration shown in FIG. 7R, the second segment 6440 is substantially perpendicular to the central axis of the central hole 6410 in the deactivated configuration. The first rotatable connection in combination with the shape of the first 6420 and second 6430 segments may make it easier for the AAV member 6400 to move between the activated and deactivated configurations. The first rotatable connection in combination with the shape of the first 6420 and second 6430 segments provides an additional degree of freedom and allows for movement of the second segment 6440 and foot portion 6450 in a direction perpendicular to the central axis. When moving between the deactivated and activated configurations the second segment 6440 and foot portion 6450 may therefore move in both a direction parallel to the central axis 6410 and perpendicular to the central axis 6410. This is advantageous in enabling the foot portion 6450 to be able to stably adopt a plurality of positions, particularly in the deactivated configuration, and may therefore increase the reliability of the AAV member 6400 to form a seal with the vent member 6200 compared to an AAV member which may only stably toggle components between two positions. The configuration of the first 6420 and second 6430 segments, for instance their positions about the first and second rotatable connections, may also depend on the arrangement and position of the surfaces against which the AAV member 6400 contacts the vent member 6200 in the deactivated configuration and the base member 6100 in the activated configuration.

Varying the design of one or more features of the AAV member 6400 may be used to vary the activation and deactivation pressure of the gas flow control structure 6000 as required for specific respiratory therapy systems.

In some forms, the greater the thickness of the first segment 6430 and second segment 6440, the greater the deactivation pressure. In some forms, the thickness of the first segment 6430 and second segment 6440 may be between 0.1 mm and 0.5 mm, in some forms the thickness is 0.17 cm. The thickness of the first segment 6430 and second segment 6440 is preferably not too small as it will increase the difficulty in manufacturing, for instance moulding, the AAV member 6400 as well as increase the risk of damage to the AAV member 6400, for instance tears or rips.

In some forms, the greater the distance between the second rotatable connection and the mounted part 6420 along a direction parallel to the central axis of the central hole 6410 when the AAV member 6400 is in a ‘neutral’ configuration, the greater the deactivation pressure. In some forms, the distance between the second rotatable connection and the mounted part 6420 along the central axis of the central hole 6410 when the AAV member 6400 is in a ‘neutral’ configuration, may be between 0.5 mm and 3 mm, in some forms the distance is 1.75 cm. This distance is preferably not too small, otherwise it will impact the range of motion of the AAV member 6400. If this distance is too small, the AAV member 6400 may get stuck in the deactivated configuration. In some forms, the greater the distance E, as shown in FIGS. 6I and 7I, between the second rotatable connection and the mounted part 6420 along a direction parallel to the central axis of the central hole 6410 when the AAV member 6400 is in the activated configuration, the greater the deactivation pressure.

In some forms, the greater the distance between the second rotatable connection and the mounted part 6420 along a direction perpendicular to the central axis of the central hole 6410 when the AAV member 6400 is in a ‘neutral’ configuration, the greater the deactivation pressure. In some forms, the distance between the second rotatable connection and the mounted part 6420 perpendicular to the central axis of the central hole 6410 may be above 0 mm and below 2 mm, in some forms the distance is 0.2 cm, in other forms the distance is 1.3 mm. In some forms, the greater the distance F, as shown in FIGS. 6I and 7I, between the second rotatable connection and the mounted part 6420 perpendicular to the central axis of the central hole 6410 when the AAV member 6400 is in the activated configuration, the greater the deactivation pressure.

In some forms, the greater the mass of the foot portion 6450, the lower the activation pressure.

In some forms, the position of the first rotatable connection on the mounted part 6420 may also affect the function of the AAV member 6400. In some forms, as shown in FIGS. 6A-7R, the first rotatable connection is positioned proximate an outer surface of the AAV member 6400, where the outer surface faces away from the first volume and towards the second volume in use. In some forms, the first rotatable connection is positioned on the outer surface of the mounted part 6420 at a region closest to the central axis of the central hole 6410. In other forms, the first rotatable connection is positioned on a side surface of the mounted part 6420, the side surface being a surface of the mounted part 6420 that is closest to the central axis of the central hole 6410. The position of the first rotatable connection on the mounted part 6420 may alter the elastic forces applied to the first 6430 and second 6430 segments and therefore the activation and/or deactivation pressures.

In some forms, to hinder the AAV member 6400 from inadvertently moving to the deactivated configuration when it is not desired, for example when the RPT device 4000 is not supplying a flow of pressurised air and the patient exhales (so that the exhaled air applies a small pressure on the AAV member), the gas control flow structure 6000 may be configured so that the AAV member 6400 is not in its neutral configuration when in the activated configuration. That is, a part of the base member 6100 is positioned to prevent the AAV member 6400 fully returning to the neutral configuration. Instead, the bias of the AAV member 6400 urges the AAV member 6400 towards the neutral configuration but the part of the base member 6100 acts as a stop, preventing the AAV member 6400 reaching the neutral configuration. In such forms, the activated configuration is the configuration of AAV member 6400 when the AAV member 6400 contacts the base member 6100. In this configuration, the part of the AAV member 6400 contacting the base member 6100 (e.g. foot portion 6450) applies a force on the base member 6100 resulting from the elastic deformation of the AAV member 6400 away from its neutral configuration and its bias to return to the neutral configuration. This force acts to hinder the AAV member from 6400 inadvertently moving to the deactivated configuration when it is not desired.

### 4.4.3 Base Member

The base member 6100 may comprise one or more features for connecting the gas flow control structure 6000 to a part of the respiratory system, for instance the plenum chamber 3200. In the form of the present technology shown in FIGS. 6A-7R the base member 6100 comprises a groove 6110 on an outer peripheral edge that is configured to form a snap-fit connection with the wall of the plenum chamber 3200. In other forms the base member 6100 may be connected to the part of the respiratory system by any one of glue, a clip mechanism, screw thread, or interference fit.

In some forms, the base member 6100 may be substantially annular in shape when projected on to a plane. In some forms, the central axis of the base member 6100 may align with the central axis of the central hole 6410 of the AAV member 6400. In some forms, a region of the base member 6100 around the outer circumference of the annular shape, may comprise the feature configured to connect the gas flow control structure 6000 to another part of the respiratory system.

In some forms, the base member 6100 may be substantially rigid. In some forms, the base member 6100 is formed by a relatively hard material. For example, the base member 6100 may be formed from a polycarbonate.

In some forms, the base member 6100 may comprise a portion with a shape configured to receive the mounted part 6410 of the AAV member 6400. For example, the base member 6100 may comprise an annular recess in a surface where the mounted part 6410 of the AAV member 6400 is configured to friction fit, snap-fit or otherwise connect to the annular recess. In the example shown in the figures, the annular recess is formed in a surface of the base member 6100 facing away from the first volume in use. Alternatively, the annular recess may be formed in a radially inwardly facing surface of the annular base member 6100.

In some forms, as shown in FIGS. 6A to 6R, the base member 6100 may also have a portion with a shape configured to receive a portion of the vent member 6200 such that they form a snap-fit connection. For example, the base member 6100 may comprise an annular recess or projection in a surface to which the vent member 6200 is configured to connect. In the example shown in FIGS. 6A-6R, the base member 6100 comprises an annular projection in a surface of the base member 6100 facing radially inwardly and the vent member 6200 has a complementary groove on an outer radial surface configured to interlock with the annular projection to connect the base member 6100 and vent member 6200 together. Other mechanisms to connect the vent member 6200 and base member 6100 together may be used in other forms of the technology.

The base member 6100 may also comprise an annular flange 6120 that the foot portion 6450 of the AAV member 6400 contacts when in the activated configuration. The annular flange 6120 may have a central axis aligned with the central axis of the central hole 6410 of the AAV member 6400. The annular flange 6120 may extend in a direction parallel to the central axis of the central hole 6410 and in a direction away from the first volume. In some forms, the annular flange 6120 may be formed around an inner circumference of the annular shape of the base member 6100. An outer surface 6121 of the flange 6120, which is a surface at the tip of the flange 6120 furthest from the side of the base member 6100 facing inwardly towards the first volume, may be the location where the foot portion 6450 contacts the annular flange 6120 in the activated configuration. The outer surface 6121 may be sloped with respect to the central axis of the central opening 6421. That is, due to the annular shape of the flange 6120, the outer surface 6121 may form a frusto-conical surface. The slope of the outer surface 6121 may correspond to the angled surface of the foot portion 6452. This may assist in ensuring the foot portion 6450 is in the correct position when in the activated configuration. In other forms the outer surface 6121 may be a substantially flat surface perpendicular to the central axis of the central opening 6421.

The base member 6100 may also comprise one or more base member openings 6130 such that gas from the first volume contacts at least a portion of the AAV member 6400. The base member openings 6130 ensure that the pressure in the first volume is applied to the AAV member 6400 such that the AAV member 6400 can move between the activated and deactivated configurations.

In some forms, the annular flange 6120 is located closer to the central axis of the central hole 6410 than the base member openings 6130. In some forms the portion of the base member 6100 configured to receive the mounted part 6420 is further from the central axis of the central hole 6410 than the base member openings 6130. That is, the base member openings 6130 may be formed in a region of the base member 6100 located between the annular flange 6120 and the portion configured to receive the mounted part 6420 of the AAV member 6400. This region may be annular in shape and extend around the central hole formed by the annular shape of the base member 6100. In this position, gas in the first volume can pass through the openings 6130 to contact the inside surface of the AAV member 6400 such that the pressure of gas in the first volume can act on the AAV member 6400.

### 4.4.4 Vent Member

In the forms of the technology shown in FIGS. 6A-7R, the vent member 6200 comprises multiple vent openings 6210 in the form of cylindrical holes through the vent member 6200, as shown in FIGS. 6A, 6D, 6J, 6M, 7A, 7D, 7J, and 7M.

In some forms, the vent member 6200 may be substantially annular in shape when projected on to a plane. In other forms, the vent member 6200 may be substantially circular in shape when projected on to a plane.

The vent member 6200 may comprise features to enable the vent member 6200 to be connected to the base member 6100. For example, as described above, the vent member 6200 may comprise a groove on an outer radial surface that is complementary to, and configured to interlock with, the annular projection of the base member 6100 to connect the base member 6100 and vent member 6200 together.

The vent member 6200 may comprise an annular flange 6220 that the foot portion 6450 of the AAV member 6400 contacts when in the deactivated configuration. The annular flange 6220 assists in forming a good seal between the foot portion 6450 and the vent member 6200 in the deactivated configuration. The annular flange 6220 may have a central axis aligned with the central axis of the central hole 6410 of the AAV member 6400. The annular flange 6220 may extend outwardly from a surface of the vent member 6200 facing the first volume in use and in a direction parallel to the central axis of the central hole 6410. An inner surface 6221, which is a surface at the tip of the flange 6220, may be the location where the foot portion 6450 contacts the annular flange 6220 in the deactivated configuration. The region of contact between the foot portion 6450 and the annular flange 6220 (i.e. the sealing surface) may lie on a single plane perpendicular to the central axis of the central hole 6410. For example, in the illustrated forms, the region of contact is an annular region at the tip of the flange 6220 lying in a plane. This may enable the AAV member 6400 to form a good seal with the vent member 6200 in the deactivated configuration, and more easily than if the sealing surface had a more complex configuration. The outer surface 6221 may be curved, for instance it may have a substantially semi-circular cross-sectional shape.

The annular flange 6120 of the base member 6100 and the annular flange 6220 of the vent member 6200 may be substantially similar in size and may be offset along the central axis of the gas flow control structure 6000. The gap between the annular flange 6120 of the base member 6100 and the annular flange 6220 of the vent member 6200 may form the AAV opening 6300. In some aspects, the distance between the annular flange 6120 of the base member 6100 and the annular flange 6220 of the vent member 6200 is between 3 mm and 6 mm, in some forms the distance is between 4 mm and 5 mm. In some forms, the total surface area of the AAV opening 6300 is between 100 mm² and 300 mm², in some forms the area is approximately 170 mm². In some forms, altering the position of the gas flow control structure 6000 within the respiratory therapy system alters the required total surface area of the AAV opening 6300 for adequate carbon dioxide washout. In some forms, the gas flow control structure 6000 is ideally located as close as possible to the patient's airways, for instance the patient's mouth, to allow for a smaller required total surface area of the AAV opening 6300 for adequate carbon dioxide washout. In general, as the path to the second volume from the patient's airways becomes more tortuous, for instance by becoming longer, the greater the total surface area of the AAV opening 6300 needs to be to allow for adequate carbon dioxide washout. In some forms, the gas flow control structure 6000 is located in a wall of the plenum chamber 3200 in front of the mouth of the patient 1000. Having a smaller total surface area of the AAV opening 6300 may have advantages in allowing the gas flow control structure 6000 to be more compact and less obtrusive for the patient 1000.

In the aspect shown in FIGS. 6A to 6R, the vent member 6200 connects directly to the base member 6100 and further comprises one or more ambient openings 6240 through which gas flows to the second volume after passing through the AAV opening 6300.

### 4.4.5 Cover Member

In some forms, as shown in FIGS. 7A to 7R, the gas flow control structure further comprises a cover member 6500. The cover member 6500 may connect to the vent member 6200 and the base member 6100. The cover member 6500 may connect to the vent member 6100 and the base member by a snap-fit connection. In other forms the connections may be achieved by any one or more of an adhesive, a clip mechanism, screw thread, or interference fit.

In some forms, the vent member 6200 may be connected to the cover member 6500 in a radially central region of the cover member 6500. The cover member 6500 may comprise a projection that extends in a direction towards the first volume and fits within a corresponding opening in the vent member 6200. The projection may snap-fit into the opening in the vent member 6200. The projection and opening may be aligned with the central axis of the central hole 6410.

In some forms, the cover member 6500 comprises one or more cover openings 6510 through which gas flows after passing through the AAV opening 6300. Gas also flows through the cover openings 6510 after passing through the vent opening 6210. The size of the cover openings 6510 are related to the amount of carbon dioxide washout. The surface area of the cover openings 6510 may total between 100 and 350 mm² and may for example be about 200 mm². The surface area of the cover openings 6510 may be greater than or approximately equal to the total surface area of the AAV opening 6300. In different forms of the technology, the surface area of the cover openings 6510 may be smaller if the flow path from the patient's airways to the second volume is less tortuous, as discussed above in relation to the surface area of the AAV opening 6300. The cover openings may be located proximate an outer periphery of the cover member 6500, for example arranged evenly spaced around the whole of the outer periphery. The cover openings may be holes in the cover member 6500 that are wholly surrounded by the cover member 6500 or notches in the outer periphery of the cover member 6500.

In the aspect shown in FIGS. 7A-7R, the cover member 6500 has a substantially dome-shape. The cover openings 6510 may be located in the cover member 6500 in a region at the base of the dome-shape. The cover openings 6510 may be equally spaced around the base of the dome-shape and may be of a size large enough to allow for effective carbon dioxide washout during use, as discussed above in relation to the AAV opening 6300.

In other aspects, the cover member 6500 may have a shape that integrates with that of the part of the respiratory system surrounding the gas flow control structure 6000, for instance the wall of the plenum chamber 3200.

The form of the gas flow control structure 6000 comprising a cover member 6500 may have a reduced size when compared to a form of the technology without a cover member 6500, particularly a reduced diameter across the width of the generally annular or circular components. For instance, when compared to the form of the technology shown in FIGS. 6A to 6R, where both designs have substantially the same surface area for the AAV openings 6300, the form comprising the cover member 6500 may be smaller. The smaller size may be a smaller circumference and diameter of the annular shape of the base member 6100. The reduced size provides several advantages including a reduced material cost and being less obtrusive for the patient. The reduced size may improve the aesthetics of the gas flow control structure 6000, since the AAV openings 6300 are more discreet. This may assist in reducing the medical-like appearance of a patient interface 3000 comprising the gas flow control structure 6000, which can be a deterrent for some patients.

The position of the cover openings 6510 on the sides of the dome-shape may also help the flow of gas exiting the gas flow control structure 6000 to flow in a direction parallel to the walls of the surrounding component rather than perpendicular (in the illustrated form, this direction is substantially perpendicular to the central axis of the AAV member 6400). This may reduce the amount of gas that may flow towards the patient's bed partner and also allows the module to be a smaller size compared to forms where the cover openings 6510 are located in a region of the cover member 6500 where they are oriented so that gas exits the gas flow control structure 6000 in a direction that is parallel to, or has a large component of flow that is parallel to, the central axis of the AAV member 6400.

In the form shown in FIGS. 7A-7R, the vent member 6200 comprises a curved surface 6250 positioned downstream of the AAV opening 6300 in use. In the illustrated form, the curved surface 6250 is formed on an inner surface of the vent member 6200 positioned closer to the first volume and on a radially outer region of the vent member 6200 furthest from the central axis of the central opening 6410. This curved surface 6250 assists in directing the flow of gas from the AAV opening 6300 through the cover openings 6510.

### 4.4.6 Diffuser

In some forms, a diffuser 6600 may be positioned adjacent the at least one vent opening 6210 so the flow of gas through the at least one vent opening 6210 passes through the diffuser 6600 prior to reaching the second volume. The diffuser 6600 may assist in diffusing the flow of gas to prevent the gas exiting the gas flow control structure 6000 jetting on the patient 1000 or bed partner 1100 and causing discomfort. The diffuser 6600 may be substantially annular in shape when projected on to a plane and may have a size smaller, for instance a smaller diameter, than the vent member 6200. The diffuser 6600 may be located adjacent an outer surface of the vent member 6200 facing away from the first volume.

In the form shown in FIGS. 6A to 6R, the diffuser 6600 comprises a diffuser cover 6610 separate to the vent member 6200 that covers the top of the diffuser material. There is a gap 6620 between the diffuser cover 6610 and the vent member 6200 through which gas exiting the diffuser 6600 flows into the second volume. The diffuser cover 6610 may connect to the vent member 6200 by a snap-fit connection. In other forms the connection means may be any one of glue, a clip mechanism, screw thread, or interference fit. In other aspects the diffuser cover 6610 may be integrally formed with the vent member 6200.

In the form shown in FIGS. 7A to 7R, there is no separate diffuser cover 6610. The cover member 6500 also covers the diffuser 6600. This allows the gas flow control structure 6000 to have a smaller size.

The vent member 6200 may also comprise one or more raised portions on an outer surface of the vent member facing away from the first volume, to offset the diffuser 6600 from the vent opening 6210 to maintain a bypass path for the flow of gas to reach the second volume in case the diffuser 6600 is blocked.

### 4.4.7 Location of the Gas Flow Control Structure

In certain forms of the technology, the gas flow control structure 6000 is located within the respiratory therapy system to separate the pressurised first volume from the surrounding ambient air, or second volume. In certain forms, the gas flow control structure 6000 is positioned sufficiently close to the patient's airways to adequately prevent carbon dioxide rebreathing.

In some forms, particularly forms where the patient interface 3000 comprises conduit headgear as described above, a patient interface 3000 comprising a plenum chamber 3200 and a seal-forming structure 3100 may also comprise a gas flow control structure 6000. The gas flow control structure 6000 may be located in a wall of the plenum chamber 3200. In some forms it may be located in an anterior wall of the plenum chamber 3200 and in a central location. Locating the gas flow control structure 6000 in such a position may be particularly advantageous since it is then located directly in front of the patient's airways during use. This allows the gas flow control structure 6000 to effectively washout carbon dioxide breathed out by the patient 1000. Patient interfaces 3000 comprising conduit headgear are particularly suited to locating the gas flow control structure 6000 in such a position as they usually do not have a connection for the air circuit 4170 in their central anterior regions. In other forms, the gas flow control structure 6000 may be located in any one of: the elbow structure, the connection member, or along the air circuit 4170.

In some forms, the AAV member 6400 of the gas flow control structure 6000 is configured to allow gas to flow between the plenum chamber 3200 and the air circuit 4170 in both the activated and deactivated configurations. The illustrated forms of the AAV member 6400 do not separate these or any of the internal pressurised volumes of the respiratory system.

In certain forms the gas flow control structure 6000 may be a modular assembly and the respiratory therapy system may be configured so that the gas flow control structure 6000 can be interchangeably connected to different respiratory therapy systems and parts thereof. For example, a respiratory therapy system may comprise openings in any one or more of the plenum chamber 3200, elbow structure, connection member, air circuit, and conduit headgear tubes, each of which are able to receive a gas flow control structure 6000 of the type described herein. Plugs may also be provided to seal the holes in which the gas flow control structure 6000 is not positioned. In this way the patient and/or their clinician is/are able to elect the position of the gas flow control structure 6000 within the respiratory therapy system that best suits the patients clinical and comfort needs.

### 4.5 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 4.5.1 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 4.6 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

### 4.7 Humidifier

### 4.7.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.8 Respiratory Therapy Modes

Various respiratory therapy modes may be implemented by the disclosed respiratory therapy system.

### 4.9 Glossary

For the purposes of the present technology disclosure, in certain forms of the present technology, one or more of the following definitions may apply. In other forms of the present technology, alternative definitions may apply.

### 4.9.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g−f/cm² and hectopascal. 1 cmH₂O is equal to 1 g−f/cm² and is approximately 0.98 hectopascal (1 hectopascal=100 Pa=100 N/m²=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.9.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240

Polycarbonate: a thermoplastic polymer of Bisphenol-A Carbonate.

### 4.9.2 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.11 Reference Signs List

1000 Patient
1100 Bed partner
3000 Patient interface
3100 Seal-forming structure
3200 Plenum chamber
3300 Positioning and stabilising structure
3400 Vent
3600 Connection port
3700 Forehead support
3800 Cannula
3810a Nasal prongs
3810b Nasal prongs
3820a Lumens
3820b Lumens
4000 RPT device
4010 External housing
4012 Upper portion
4014 Lower portion
4015 Panel(s)
4016 Chassis
14018 Handle
4020 Pneumatic block
4100 Pneumatic components
4110 Air filters
4112 Inlet air filter
4114 Outlet air filter
4120 Muffler
4122 Inlet muffler
4124 Outlet muffler
4140 Pressure generator
4142 Blower
4144 Brushless DC motor
4160 Anti-spill back valve
4170 Air circuit
4180 Supplemental oxygen
4200 Electrical components
4202 Printed Circuit Board Assembly (PCBA)
4210 Power Supply
4220 Input devices
4270 Transducers
5000 Humidifier
5002 Humidifier inlet
5004 Humidifier outlet
5006 Humidifier base
5110 Reservoir
5120 Conductive portion
5130 Humidifier reservoir dock
5135 Locking lever
5150 Water level indicator
5240 Heating element
6000 Gas flow control structure
6100 Base member
6110 Groove
6120 Annular flange of base member
6121 Outer surface of annular flange of base member
6130 Base member openings
6200 Vent member
6210 Vent opening
6220 Annular flange of vent member
6221 Inner surface of annular flange of vent member
6240 Ambient openings of vent member
6250 Curved surface
6300 AAV opening
6400 AAV member
6410 Central hole
6420 Mounted part
6430 First segment
6431 First end of first segment
6432 Second end of first segment
6440 Second segment
6441 First end of second segment
6442 Second end of second segment
6450 Foot portion
6451 Flat surface of foot portion
6452 Angled surface of foot portion
6500 Cover member
6510 Cover opening
6600 Diffuser
6610 Diffuser cover
6620 Gap between diffuser cover and vent member

1. A gas flow control structure for a respiratory therapy system configured to function as both an anti-asphyxia valve (AAV) and a vent, wherein the gas flow control structure separates a first volume that is pressurised during use and a second volume that is the surrounding ambient air, the gas flow control structure comprising:
a base member;
a vent member comprising at least one vent opening through which a flow of gas can pass from the first volume to the second volume during use;
an AAV opening formed between the base member and the vent member; and
an AAV member mounted to the base member, wherein the AAV member is a flexible, resilient membrane which is substantially annular in shape when projected on to a plane, the annular shape of the AAV member forming a central hole,
wherein the AAV member is movable between: an activated configuration in which gas can flow between the first volume and the second volume through the AAV opening; and a deactivated configuration in which the AAV member forms a seal with a portion of the vent member so gas is blocked from flowing through the AAV opening,
wherein the AAV member is biased towards the activated configuration such that, when the first volume contains gas having a pressure that is below an activation pressure, the AAV member is in the activated configuration, and, when the first volume contains gas having a pressure that is above a deactivation pressure, the AAV member is in the deactivated configuration;
wherein the AAV member is arranged such that gas from the first volume flows through the central hole in the AAV member when passing to the second volume through the at least one vent opening.

2. The gas flow control structure of claim 1, wherein the AAV member comprises:
a first segment with a first end and an opposite second end, wherein the first end of the first segment is rotatably connected to a mounted part of the AAV member at a first rotatable connection, wherein the mounted part of the AAV member is mounted to the base member;
a second segment with a first end and an opposite second end, wherein the first end of the second segment is rotatably connected to the second end of the first segment at a second rotatable connection.

3. The gas flow control structure of claim 2, wherein the AAV member further comprises a foot portion that is rotatably connected to the second end of the second segment at a third rotatable connection.

4. The gas flow control structure of claim 3, wherein the foot portion contacts a portion of the base member in the activated configuration.

5. The gas flow control structure of claim 4, wherein the base member comprises an annular flange that the foot portion contacts in the activated configuration.

6. The gas flow control structure of claim 3, wherein the foot portion forms the seal with the portion of the vent member in the deactivated configuration.

7. The gas flow control structure of claim 6, wherein a substantially flat plane of the foot portion forms the seal with the portion of the vent member in the deactivated configuration.

8. The gas flow control structure of claim 6, wherein the vent member comprises an annular flange that the foot portion forms the seal with in the deactivated configuration.

9. The gas flow control structure of claim 8, wherein the annular flange of the vent member and the annular flange of the base member are substantially similar in size and are offset along a central axis of the gas flow control structure.

10. The gas flow control structure of claim 1, further comprising a diffuser positioned adjacent the at least one vent opening so the flow of gas through the at least one vent opening passes through the diffuser prior to reaching the second volume.

11. The gas flow control structure of claim 1, further comprising a cover member, wherein the cover member connects to the vent member and to the base member, and the cover member comprises one or more cover openings through which gas flows after passing through the AAV opening and after passing through the vent opening.

12. The gas flow control structure of claim 11, wherein the cover member is substantially dome-shaped.

13. The gas flow control structure of claim 1, wherein the vent member connects directly to the base member and further comprises one or more ambient openings through which gas flows after passing through the AAV opening.

14. The gas flow control structure of claim 13, further comprising a diffuser cover to cover the diffuser, wherein a gap between the vent member and the diffuser cover allows gas to flow from the diffuser to the second volume.

15. The gas flow control structure of claim 1, wherein a neutral configuration of the AAV member is different to the activated configuration.

16. A patient interface comprising:
a plenum chamber pressurisable to a therapeutic pressure of at least 6 cmH2O above ambient air pressure, said plenum chamber including a plenum chamber inlet port sized and structured to receive a flow of gas at the therapeutic pressure for breathing by a patient;
a seal-forming structure constructed and arranged to form a seal with a region of the patient's face surrounding an entrance to the patient's airways, said seal-forming structure having a hole therein such that the flow of gas at said therapeutic pressure is delivered to at least an entrance to the patient's nares, the seal-forming structure constructed and arranged to maintain said therapeutic pressure in the plenum chamber throughout the patient's respiratory cycle in use; and
a gas flow control structure according to claim 1, wherein the gas flow control structure allows a continuous flow of gas exhaled by the patient from an interior of the plenum chamber to ambient, said gas flow control structure being sized and shaped to maintain the therapeutic pressure in the plenum chamber in use when the AAV member is in the deactivated configuration, and wherein the gas flow control structure allows the patient to inhale and exhale air from ambient when the flow of gas at the therapeutic pressure is not being supplied to the plenum chamber.

17. The patient interface of claim 16, wherein the gas flow control structure is located in a wall of the plenum chamber.

18. A respiratory therapy system comprising:
a patient interface according to claim 16,
at least one air circuit for supplying the flow of gas at the therapeutic pressure to the patient interface.

19. The respiratory therapy system of claim 18, wherein the AAV member is configured to allow gas to flow between the plenum chamber and the air circuit in both the activated configuration and deactivated configuration.
